(19) United States
(12) Patent Application Publication
(10) Pub. No.: US 2015/0353631 A1
(43) Pub. Date: Dec. 10, 2015

(54) C-TERMINAL AND CENTRAL EPITOPE A-BETA ANTIBODIES

(71) Applicants: JANSSEN ALZHEIMER IMMUNOTHERAPY, Dublin (IE); WYETH LLC, Madison, NJ (US)

(72) Inventors: Manuel Buttini, Emeryville, CA (US); Ming Chen, Hayward, CA (US); Shyra Gardai, Hayward, CA (US)

(21) Appl. No.: 14/410,018

(22) PCT Filed: Jun. 18, 2013
(86) PCT No.: PCT/US13/46399

§ 371 Date: Dec. 19, 2014

Related U.S. Application Data
(60) Provisional application No. 61/667,891, filed on Jul. 3, 2012.

(51) Int. Cl. C07K 16/18; A61K 39/395; A61K 49/00

(52) U.S. Cl. CPC C07K 16/18; A61K 49/00; A61K 39/3955; C07K 2317/24; C07K 2317/565; A61K 2039/507

(57) ABSTRACT

The present invention provides antibodies directed against C-terminal and central epitopes of Aβ that preferentially bind compact plaques relative to diffuse plaques. The invention also provides methods of treating patients to reduce or eliminate the presence of compact plaques of Aβ and associated symptoms.

### CROSS-REFERENCE TO RELATED APPLICATION

This application claims the benefit under 35 USC 119(e) of U.S. Provisional Patent Application No. 61/667,891, filed Jul. 3, 2012, which is hereby incorporated by reference in its entirety.

### BACKGROUND

Alzheimer's disease (AD) is a progressive disease resulting in senile dementia. See generally Selkoe, TINS 16:403 (1993); Hardy et al., WO 92/13069; Selkoe, J. Neuropathol. Exp. Neurol. 53:438 (1994); Duff et al., Nature 373:476 (1995); Games et al., Nature 373:523 (1995). Broadly speaking, the disease falls into two categories: late onset, which occurs in old age (65+ years) and early onset, which develops well before the senile period, i.e., between 35 and 60 years. In both types of disease, the pathology is the same but the abnormalities tend to be more severe and widespread in cases beginning at an earlier age. The disease is characterized by at least two types of lesions in the brain, neurofibrillary tangles and senile plaques. Neurofibrillary tangles are intracellular deposits of microtubule associated tau protein consisting of two filaments twisted about each other in pairs. Senile plaques (i.e., amyloid plaques) are areas of disorganized neuropile up to 150 μm across with extracellular amyloid deposits at the center which are visible by microscopic analysis of sections of brain tissue. The accumulation of amyloid plaques within the brain is also associated with Down's syndrome and other cognitive disorders.

The principal constituent of the plaques is a peptide termed Aβ or β-amyloid peptide. Aβ is a 4-kDa internal fragment of 39-43 amino acids of a larger transmembrane glycoprotein named amyloid precursor protein (APP). As a result of proteolytic processing of APP by different secretase enzymes, Aβ is primarily found in both a short form, 40 amino acids in length, and a long form, ranging from 42-43 amino acids in length. Part of the hydrophobic transmembrane domain of APP is found at the carboxy end of Aβ, and may account for the ability of Aβ to aggregate into plaques, particularly in the case of the long form. Accumulation of amyloid plaques in the brain eventually leads to neuronal cell death. The physical symptoms associated with this type of neural deterioration characterize Alzheimer's disease.

The presence of plaques in AD brains is most reliably revealed using immunostaining techniques with Aβ specific antibodies (Hyman et al., Proc Natl Acad Sci USA 92:3586-3590 (1995)). Two of the most commonly used antibodies (3D6 and 10D5) recognize N-terminal Aβ epitopes (within residues 1-5 and 3-7 respectively) (Hyman et al. (1995), supra; Bard et al., Proc Natl Acad Sci USA 100:2023-2028 (2003)).

Some antibodies against the C-terminal part of Aβ have been reported to bind to amyloid deposits. However, such studies have typically used formalin- or paraformaldehyde-fixed, paraffin embedded tissues that are often subjected to aggressive pre-treatments with formic acid or other aggressive reagents to reveal the C-terminal epitopes of Aβ (Murphy et al., Am J Pathol 144:1082-1088 (1994); Kida et al., Neurosci Let 193:105-108 (1995); Fukumoto et al., Am J Pathol 148:259-265 (1996); Mann et al., Am J Pathol 148:1257-1266 (1996); Tekirian et al., Neurobiol Aging 17:249-257 (1996); Lippa et al., Arch Neurol 56:1111-1118 (1999); Schwab et al., Exp Neurol 161:527-534 (2000); Axelsen et al., Mol Immunol 46:2267-2273 (2009)). Thus, these studies may not be relevant to physiologically plaques. Wilcock et al., J. Neurosci 26:5340-5346 (2006) and Wilcock et al., J Neurosci 24:6144-6151 (2004) reported reduction of plaques in Tg2576 mice receiving injections of a 2H6 antibody (anti-Aβ₃₃₋₄₀; end-specific, mouse IgG2b isotype) or a 2286 antibody (anti-Aβ₂₈₋₄₀; end-specific, mouse IgG1 isotype), respectively. Several other C-terminal antibodies including 16C11, 2G3, and 21F12 have been reported to be unable to bind or clear plaques in a PDAPP animal model of Alzheimer's disease (see U520060257396).

The 266 antibody (anti-Aβ₁₆₋₂₃) has been reported to bind predominantly to soluble forms of Aβ and to shows little binding to plaques. Such studies have also reported conflicting results regarding the ability of this antibody to clear plaques from the brains of treated mice. DeMattos et al. (Proc Natl Acad Sci USA 98:8850-8855 (2001)) report that a 266 antibody peripherally administered to PDAPP mice clears plaques without binding to the plaques or entering the brain, but rather by capturing soluble Aβ in the periphery and decreasing brain plaque load by producing a concentration gradient by which Aβ is shifted away from the brain and into the plasma (peripheral sink hypothesis). However, a later study in PDAPP mice immunized with the 266 antibody reported no reduction in plaques (Seubert et al., Neurodegener Dis 5:65-71 (2008)). A third study reported that short-term treatment of PDAPP mice with the 266 antibody reverses their cognitive deficits without affecting plaque, proposing that the 266 antibody binds and neutralizes soluble neurotoxic Aβ species from the brain (Dodart et al., Nat Neurosci 5:452-457 (2002)). 0520060257396 reports that the 266 antibody and two other antibodies binding to mid-region epitopes in Aβ (18G11 and 22D12) neither bound nor cleared plaques in the PDAPP transgenic animal model.

### SUMMARY OF THE INVENTION

The present invention provides antibodies directed against C-terminal and central epitopes of Aβ that preferentially bind compact plaques relative to diffuse plaques. The invention also provides methods of treating patients to reduce or eliminate the presence of compact plaques of Aβ and associated symptoms.

In one aspect, the present invention provides a method of treating a patient diagnosed with mid- or late-stage Alzheimer's disease, comprising administering to the patient an effective regime of an antibody that binds to an epitope within residues 12-43 of Aβ and preferentially binds compact plaques relative to diffuse plaques. In some cases, the patient has been diagnosed with mid-stage Alzheimer's disease. In some cases, the patient has been diagnosed with late-stage Alzheimer's disease.

In another aspect, the present invention provides a method of treating a patient diagnosed with Alzheimer's disease and having a greater proportion of compact plaques relative to diffuse plaques, comprising administering to the patient an effective regime of an antibody that binds to an epitope within residues 12-43 of Aβ and preferentially binds compact plaques relative to diffuse plaques. In some cases, the proportion of compact plaques is at least 40% of total plaques. In some cases, the proportion of compact plaques relative to diffuse plaques is determined by positron emission tomography (PET) scanning. In some cases, the PET scanning comprises detecting a PET ligand selected from the group consisting of [¹⁸F]AV-14, [¹⁸F]AV-144, [¹¹C]AZD2995, [¹⁸F]-AZD4694 and [¹⁸F]-SMIBR-W372.

In another aspect, the present invention provides a method of treating a patient diagnosed with Alzheimer's disease and having symptoms of epileptic seizures, comprising administering to the patient an effective regime of an antibody that binds to an epitope within residues 12-43 of Aβ and preferentially binds compact plaques relative to diffuse plaques. In some cases, total amyloid plaque burden and the symptoms of epileptic seizures are reduced.

In another aspect, the present invention provides a method of treating a patient diagnosed with Alzheimer's disease, comprising: (a) administering to the patient an effective regime of an antibody that preferentially binds compact plaques relative to diffuse plaques, wherein the antibody has specificity for a central or C-terminal epitope of Aβ; and (b) monitoring one or more attributes of compact plaques in the patient's brain using PET scanning. In some cases, the one or more attributes of the compact plaques is identified using radiotracer PiB. In some cases, the one or more attributes comprise a reduction in size of one or more compact plaques relative to a prior PET scan.

In another aspect, the present invention provides a method of treating a patient diagnosed with Alzheimer's disease that has previously been treated with an antibody with specificity for an N-terminal epitope of Aβ, comprising administering to the patient an effective regime of an antibody that binds to an epitope within residues 12-43 of Aβ and preferentially binds compact plaques relative to diffuse plaques. In some cases, the patient's proportion of compact plaques relative to total plaques increased during prior treatment with the antibody specific for an N-terminal epitope of Aβ.

In another aspect, the present invention provides a method of treating a patient diagnosed with Alzheimer's disease that has previously been treated with an antibody that binds to an epitope within residues 12-43 of Aβ and preferentially binds compact plaques relative to diffuse plaques, comprising administering to the patient an effective regime of an antibody with specificity for an N-terminal epitope of Aβ. In some cases, the patient's proportion of diffuse plaques relative to total plaques increased during prior treatment with the antibody specific for a central or C-terminal epitope of Aβ.

In another aspect, the present invention provides a method of treating a patient diagnosed with Alzheimer's disease, comprising: (a) administering to the patient an effective regime of an antibody that binds to an epitope within residues 12-43 of Aβ and preferentially binds compact plaques relative to diffuse plaques; and (b) administering to the patient an effective regime of a second antibody with specificity for an N-terminal epitope of Aβ. In some cases, the first and second antibodies are administered concurrently. In some cases, the second antibody is selected from a 3D6 antibody, a 12A11 antibody, a 10D5 antibody, a 12B4 antibody, a 6C6 antibody, a 2H3 antibody, or a 3A3 antibody, or a chimeric, humanized or veneered form of any one of these antibodies.

In various embodiments of any one of the methods discussed in the preceding paragraphs, the antibody has specificity for a central epitope of Aβ, or the antibody has specificity for a C-terminal epitope of Aβ.

In some cases, the antibody has a specificity for a central epitope of Aβ, and the antibody is a 266 antibody or a chimeric, humanized or veneered form thereof, a 15C11 antibody or a chimeric, humanized or veneered form thereof, or a 22D12 antibody or a chimeric, humanized or veneered form thereof. In some cases, the antibody comprises: three light chain variable region complementarity determining regions (CDRs), wherein CDR L1 comprises the amino acid sequence of SEQ ID NO:4, CDR L2 comprises the amino acid sequence of SEQ ID NO:5, and CDR L3 comprises the amino acid sequence of SEQ ID NO:6, and three heavy chain variable region CDRs, wherein CDR H1 comprises the amino acid sequence of SEQ ID NO:7, CDR H2 comprises the amino acid sequence of SEQ ID NO:8, and CDR H3 comprises the amino acid sequence of SEQ ID NO:9. In some cases, the antibody comprises: three light chain variable region CDRs, wherein CDR L1 comprises the amino acid sequence of residues 24 to 39 of SEQ ID NO:14, CDR L2 comprises the amino acid sequence of residues 55 to 61 of SEQ ID NO:14, and CDR L3 comprises the amino acid sequence of residues 94 to 101 of SEQ ID NO:14, and three heavy chain variable region CDRs, wherein CDR H1 comprises the amino acid sequence of residues 26 to 35 of SEQ ID NO:15, CDR H2 comprises the amino acid sequence of residues 50 to 66 SEQ ID NO:15, and CDR H3 comprises the amino acid sequence of residues 99 to 101 of SEQ ID NO:15. In some cases, the antibody comprises: three light chain variable region CDRs of 22D12, and three heavy chain variable region CDRs of 22D12.

In some cases, the antibody has a specificity for a C-terminal epitope of Aβ, and the antibody is a 2G3 antibody or a chimeric, humanized or veneered form thereof, a 14C2 antibody or a chimeric, humanized or veneered form thereof, or a 21F12 antibody or a chimeric, humanized or veneered form thereof. In some cases, the antibody comprises: three light chain variable region complementarity determining regions (CDRs) of 2G3, and three heavy chain variable region CDRs of 2G3. In some cases, the antibody comprises: three light chain variable region CDRs of 14C2, and three heavy chain variable region CDRs of 14C2. In some cases, the antibody comprises: three light chain variable region CDRs, wherein CDR L1 comprises the amino acid sequence of residues 24 to 39 of SEQ ID NO:3, CDR L2 comprises the amino acid sequence of residues 55 to 61 of SEQ ID NO:3, and CDR L3 comprises the amino acid sequence of residues 94 to 102 of SEQ ID NO:3, and three heavy chain variable region CDRs, wherein CDR H1 comprises the amino acid sequence of residues 26 to 35 of SEQ ID NO:2, CDR H2 comprises the amino acid sequence of residues 50 to 66 of SEQ ID NO:2, and CDR H3 comprises the amino acid sequence of residues 99 to 106 of SEQ ID NO:2.

In some cases, any one of the antibodies discussed above is a chimeric antibody or a humanized antibody. In a preferred embodiment, the antibody is a humanized antibody. In some cases, the antibody is of the IgG1 subtype.

In another aspect, the present invention provides a humanized, chimeric or veneered form of an antibody designated 2G3, 14C2, 21F12, or 22D12. In some cases, the antibody comprises six Kabat CDRs of the 2G3, 14C2, 21F12 or 22D12 antibody.

The invention further provides methods of treating a patient diagnosed with Alzheimer's disease and having a greater proportion of compact plaques than diffuse plaques relative to total plaques, comprising administering to the patient an effective regime of an antibody that binds to an epitope within residues 1-11 of Aβ. In some such methods, the proportion of compact plaques is at least 40% of total plaques. In some such methods, the proportions of compact and diffuse plaques relative to total plaques are determined by positron emission tomography (PET) scanning.

The invention further provides methods of treating a patient diagnosed with Alzheimer's disease and having an MMSE of 1-9 or Braak of 6-7, comprising administering to the patient an effective regime of an antibody that binds to an epitope within residues 12-43 of Aβ and preferentially binds compact plaques relative to diffuse plaques.

### BRIEF DESCRIPTION OF THE DRAWINGS

FIGS. 1A and 1B show the binding of C-terminal epitope specific antibodies 2G3, 14C2 and 21F12 to plaques in unfixed AD brain sections (occipital cortex) independent of ApoE genotype. Each row shows sections from a different patient with the apoE3/E3 genotype (FIG. 1A), or the apoE3/E4 genotype (FIG. 1B). The 3D6 antibody is a positive control. IgG, used as a negative control, showed no staining (not shown).

FIGS. 2A-2D show the relative binding of a 3D6 antibody (positive control), and C-terminal epitope specific antibodies 2G3, 14C2 and 21F12, respectively, to plaques in unfixed and fixed brain sections of PDAPP, PSAPP, and Line 41 mice. IgG, used as a negative control, showed no staining (not shown).

FIGS. 3A and 3B show immunostaining of unfixed AD brain sections (FIG. 3A) and unfixed PSAPP mouse brain sections (FIG. 3B) for 3D6, 21F12 and 2G3 antibodies, and combinations of 3D6 with 21F12 or 2G3, demonstrating that 2G3 and 21F12 antibodies bind primarily to the dense core of plaques.

FIGS. 4A and 4B show the results of two separate ex vivo experiments evaluating the induction of plaque clearance from PSAPP and Line 41 mice brain sections by IgG (negative control), 3D6 antibodies (positive control), and C-terminal epitope specific antibodies 2G3, 14C2 and 21F12. The white spots in each panel represent signals from 3D6-stained plaques.

FIG. 5 shows the induction of microglial phagocytosis of plaques from PSAPP (left panels) and Line 41 (right panels) mice brain sections in an ex vivo assay. The presence of Aβ inside microglia is visible in each of the lower six panels.

FIGS. 6A and 6B show the binding of central epitope specific antibodies 266, 15C11 and 22D12 to plaques in unfixed AD brain sections (occipital cortex) independent of ApoE genotype. Each row shows sections from a different patient with the apoE3/E3 genotype (FIG. 6A), or the apoE3/E4 genotype (FIG. 6B). The 3D6 antibody is a positive control. IgG, used as a negative control, showed no staining (not shown).

FIG. 7 shows the relative binding of a 3D6 antibody (positive control), and central epitope specific antibodies 266, 15C11 and 22D12 to plaques in unfixed PDAPP and PSAPP mice brain sections. IgG, used as a negative control, showed no staining (not shown). A background image of non-transgenic control mice (Non-Tg) is also shown.

FIGS. 8A and 8B show immunostaining of unfixed AD brain sections (FIG. 8A) and unfixed PSAPP mouse brain sections (FIG. 8B) for 3D6 and 22D12 antibodies, and combinations of the two antibodies, demonstrating that 22D12 antibodies bind primarily to the dense core of plaques.

FIG. 9 shows the results of an ex vivo experiment evaluating the induction of plaque clearance from PDAPP and PSAPP mice brain sections by IgG (negative control), 3D6 antibodies (positive control), and central epitope specific antibodies 266, 15C11 and 22D12. The white spots in each panel represent signals from 3D6-stained plaques. The central epitope specific antibodies do not significantly clear plaques in the PDAPP sections, but did clear plaques in sections of Line 41 mice.

FIG. 10 shows the induction of microglial phagocytosis of plaques from PDAPP (left panels) and PSAPP (right panels) mice brain sections in an ex vivo assay. The presence of Aβ inside microglia is visible in the both panels of the 3D6 antibody (positive control), and in the lower right panel corresponding to the 266 antibody in PSAPP mouse sections.

### BRIEF DESCRIPTION OF THE SEQUENCES

TABLE 1
Description of sequences.

| SEQ ID NO | Description of Amino Acid Sequence |
|---|---|
| 1 | Aβ 1-42 |
| 2 | m21F12 V_{H} with signal sequence |
| 3 | m21F12 V_{L} with signal sequence |
| 4 | m266 CDR L1 |
| 5 | m266 CDR L2 |
| 6 | m266 CDR L3 |
| 7 | m266 CDR H1 |
| 8 | m266 CDR H2 |
| 9 | m266 CDR H3 |
| 10 | h266 V_{L} |
| 11 | h266 V_{H} |
| 12 | h266 V_{L} |
| 13 | h266 V_{H} |
| 14 | m15C11 V_{L} |
| 15 | m15C11 V_{H} |
| 16 | bapineuzumab V_{L} |
| 17 | bapineuzumab V_{H} |
| 18 | h10D5 V_{L} |
| 19 | h10D5 V_{H} |
| 20 | h12A11 V_{L} |
| 21 | h12A11 V_{H} |

### DEFINITIONS

The basic antibody structural unit comprises a tetramer of subunits. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. The heavy chain constant region includes CH1, hinge, CH2, and CH3 domains.

Light chains are classified as either kappa or lambda. Heavy chains are classified as gamma, mu, alpha, delta, or epsilon, and define the antibody's isotype as IgG, IgM, IgA, IgD and IgE, respectively. Within light and heavy chains, the variable and constant regions are joined by a “J” region of about 12 or more amino acids, with the heavy chain also including a “D” region of about 10 more amino acids. (See generally, Fundamental Immunology (Paul, W., ed., 2nd ed. Raven Press, N.Y., 1989), Ch. 7 (incorporated by reference in its entirety for all purposes). In humans, there are four IgG isotypes, IgG1, 2, 3 and 4 Amino acids in the heavy chain constant region are number by the EU numbering convention.

The variable regions of each light/heavy chain pair form the antibody binding site. Thus, an intact antibody has two binding sites. Except in bifunctional or bispecific antibodies, the two binding sites are the same. The chains all exhibit the same general structure of relatively conserved framework regions (FR) joined by three hypervariable regions, also called complementarity determining regions or CDRs. The CDRs from the two chains of each pair are aligned by the framework regions, enabling binding to a specific epitope. From N-terminal to C-terminal, both light and heavy chains comprise the domains FRE CDR1, FR2, CDR2, FR3, CDR3 and FR4. The assignment of amino acids to each domain is preferably in accordance with the definitions of Kabat, Sequences of Proteins of Immunological Interest (National Institutes of Health, Bethesda, Md., 1987 and 1991), or Chothia & Lesk, J. Mol. Biol. 196:901-917 (1987). However, CDRs can alternatively be defined according to Chothia et al., Nature 342:878-883 (1989) or by a composite of Kabat and Chothia definitions in which any amino acid occurring within a CDR defined by Kabat or Chothia is considered part of the CDR and other residues are considered framework residues.

Reference to an antibody or immunoglobulin includes intact antibodies and binding fragments thereof. Typically, fragments compete with the intact antibody from which they were derived for specific binding to an antigen. Fragments include separate heavy and light chains, Fab, Fab′ F(ab′)2, Fabc, and Fv. Separate chains include NANOBODIES™ (i.e., the isolated VH fragment of the heavy chain of antibodies from camels or llamas, optionally humanized). Isolated VH fragments can also be obtained from other sources, such as human antibodies. Fragments are produced by recombinant DNA techniques, or by enzymatic or chemical separation of intact immunoglobulins. The term “antibody” also includes one or more immunoglobulin chains that are chemically conjugated to, or expressed as, fusion proteins with other proteins. The term “antibody” also includes bispecific antibodies. A bispecific or bifunctional antibody is an artificial hybrid antibody having two different heavy/light chain pairs and two different binding sites. Bispecific antibodies can be produced by a variety of methods including fusion of hybridomas or linking of Fab′ fragments. (See, e.g., Songsivilai & Lachmann, Clin. Exp. Immunol. 79:315-321 (1990); Kostelny et al., J. Immunol. 148, 1547-1553 (1992).)

Specific binding of a monoclonal antibody to its target antigen means an affinity of at least 10⁶, 10⁷, 10⁸, 10⁹, or 10¹⁰ M⁻¹. Specific binding is detectably higher in magnitude and distinguishable from non-specific binding occurring to at least one unrelated target. Specific binding can be the result of formation of bonds between particular functional groups or particular spatial fit (e.g., lock and key type) whereas nonspecific binding is usually the result of van der Waals forces. Specific binding does not however necessarily imply that a monoclonal antibody binds one and only one target.

An antibody that “preferentially binds” compact plaques relative to diffuse plaques is one that generates a more intense signal in an immunostaining assay (e.g., Hyman et al. (1995), supra). Such a comparison can be performed between compact and diffuse plaques in the same tissue section (e.g., from a human). Alternatively, the comparison can be performed between tissue sections containing different representations of compact plaques, for example, comparing a tissue section from a PSAPP mouse (Holcomb et al., Nat Med 4:97-100 (1998); Gordon et al., Exp Neurol 173:183-195 (2002)) with one from a PDAPP mouse having a lower representation of compact plaques (Games et al., Nature 373:523-527 (1995)). Because there may be some variation between the staining of individual plaques, whether compact or diffuse, the comparison is preferably based on an average or mean staining of several compact plaques and several diffuse plaques and the difference should be sufficient that the increased staining of compact to diffuse plaques is beyond a reasonable measure of variation of staining of diffuse plaques (e.g., mean plus a standard deviation). The immunostaining can be compared by eye or more quantitatively by digitalizing the stain to a numeric value representative of staining intensity.

The term “epitope” refers to a site on an antigen to which an immunoglobulin or antibody (or antigen binding fragment thereof) specifically binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, G. E. Morris, Ed. (1996).

Antibodies that recognize the same or overlapping epitopes can be identified in a simple immunoassay showing the ability of one antibody to compete with the binding of another antibody to a target antigen. The epitope of an antibody can also be defined X-ray crystallography of the antibody bound to its antigen to identify contact residues. Alternatively, two antibodies have the same epitope if all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Competition between antibodies is determined by an assay in which an antibody under test inhibits specific binding of a reference antibody to a common antigen (see, e.g., Junghans et al., Cancer Res. 50:1495, 1990). A test antibody competes with a reference antibody if an excess of a test antibody (e.g., at least 2×, 5×, 10×, 20× or 100×) inhibits binding of the reference antibody by at least at least 50% but preferably 75%, 90% or 99% as measured in a competitive binding assay. Antibodies identified by competition assay (competing antibodies) include antibodies binding to the same epitope as the reference antibody and antibodies binding to an adjacent epitope sufficiently proximal to the epitope bound by the reference antibody for steric hindrance to occur.

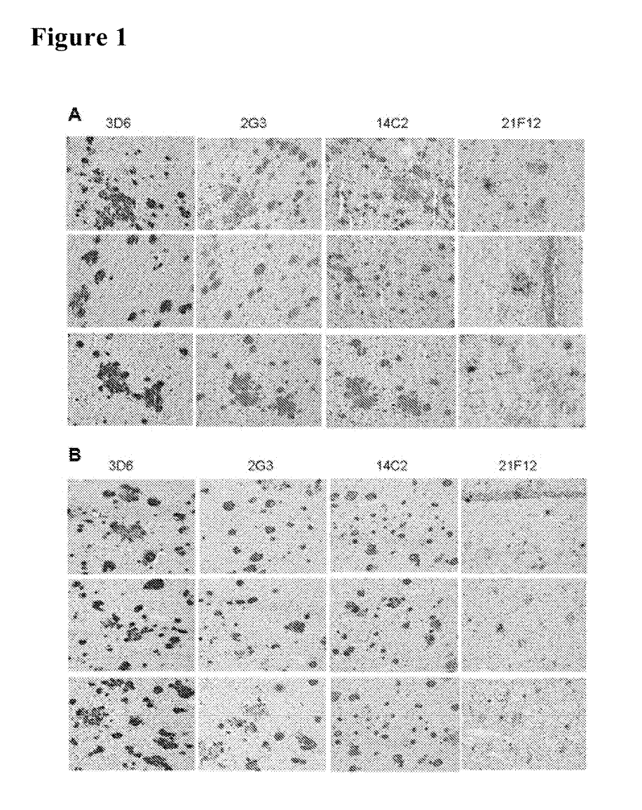

Multiple isoforms of APP exist, for example APP⁶⁹⁵, APP⁷⁵¹ and APP⁷⁷⁰. Unless otherwise apparent from the context, amino acids within APP are assigned numbers according to the sequence of the APP⁷⁷⁰ isoform (see e.g., GenBank Accession No. P05067). The sequences of Aβ peptides and their relationship to the APP precursor are illustrated by FIG. 1 of Hardy et al., TINS 20, 155-158 (1997). For example, Aβ42 has the sequence: H₂N-Asp-Ala-Glu-Phe-Arg-His-Asp-Ser-Gly-Tyr-Glu-Val-His-His-Gln-Lys-Leu-Val-Phe-Phe-Ala-Glu-Asp-Val-Gly-Ser-Asn-Lys-Gly-Ala-Ile-Ile-Gly-Leu-Met-Val-Gly-Gly-Val-Val-Ile-Ala-OH (SEQ ID NO:1).

Unless otherwise apparent from the context, reference to Aβ also includes natural allelic variations of the above sequence, particularly those associated with hereditary disease, such as the Arctic mutation, E693G, APP 770 numbering. Aβ41, Aβ40 and Aβ39 differ from Aβ42 by the omission of Ala, Ala-Ile, and Ala-Ile-Val respectively from the C-terminal end. Aβ43 differs from Aβ42 by the presence of a threonine residue at the C-terminus.

An N-terminal epitope of Aβ means an epitope within residues 1-11. An epitope within a C-terminal region means an epitope within residues 29-43, and an epitope within a central or mid-region means an epitope within residues 12-28. When an epitope occurs with a range, it can include the amino acids defining the range as well as amino acids in between. When antibodies to central and C-terminal regions are referred to collectively, such an antibody can have an epitope within a central or C-terminal region or spanning the boundary between central and C-terminal regions. That is, the epitope is within residues 12-43 of Aβ.

Monomeric Aβ and small oligomeric assemblies of about 4-10 monomers, sometimes known as ADDLs (Lambert et al., PNAS May 26, 1998 vol. 95 no. 11 6448-6453), are soluble in aqueous solution, including body fluids, such as CSF. Higher order assemblies of Aβ formed by in vitro aggregation or in vivo in the form of plaques are substantially insoluble in aqueous solutions. Aggregated Aβ is believed to be held together at least in part, by hydrophobic residues at the C-terminus of the peptide (part of the transmembrane domain of APP). Higher order insoluble deposits are sometimes referred to as amyloid fibrils. Fibrils are characterized by a cross-beta structure and are substantially insoluble even in detergents and denaturing solvents (see Schmidt et al., PNAS 106, 19813-19818 (2009); Cai et al., Current Medicinal Chemistry 24, 19-52 (2007)).

Plaques are classified by the tripartide scheme of Dickson & Vickers, Neuroscience. 2001; 105(1):99-107). The term “compact” or “fibrillar” plaque refers to plaques having a morphological phenotype of a central mass of Aβ with compact spoke-like extensions leading to a confluent outer rim (Dickson, Neuroscience. 2001; 105(1):99-107).

The term “diffuse plaque” refers to plaques lacking a morphologically identifiable substructure appearing as a substantially homogeneous sphere of Aβ.

The term “dense-cored plaque” refers to plaques having a morphological phenotype of a compacted central mass of Aβ surrounded by an outer sphere of substantially homogeneous Aβ. Because dense-cored plaques have characteristics of both diffuse and compact plaques, they can either be treated as a distinct category from compact and diffuse plaques or or allocated between these plaque classes based on the relative surface areas or volumes of compact and diffuse regions within dense-cored plaques.

The term “Fc region” refers to a C-terminal region of an IgG heavy chain. Although the boundaries of the Fc region of an IgG heavy chain can vary slightly, an Fc region is typically defined as spanning from about amino acid residue Cys226 to the carboxyl-terminus of an IgG heavy chain(s).

The term “effector function” refers to an activity that resides in the Fc region of an antibody (e.g., an IgG antibody) and includes, for example, the ability of the antibody to bind effector molecules such as complement and/or Fc receptors, which can control several immune functions of the antibody such as effector cell activity, lysis, complement-mediated activity, antibody clearance, and antibody half-life. Effector function can also be influenced by mutations in the hinge region.

The term “Kabat numbering” is defined as the numbering of the residues as in Kabat et al. (Sequences of Proteins of Immunological Interest, 5th Ed. Public Health Service, National Institutes of Health, Bethesda, Md. (1991)), incorporated herein by reference.

The term “adjuvant” refers to a compound that when administered in conjunction with an antigen elicits and/or augments an immune response to the antigen, but when administered alone does not generate an immune response to the antigen. Adjuvants can augment an immune response by several mechanisms including lymphocyte recruitment, stimulation of B and/or T cells, and stimulation of macrophages.

The term “ApoE4 carrier” refers to patients having one or two ApoE4 alleles, and the terms “ApoE4 noncarrier,” ApoE4 non-carrier” or “non-ApoE4 carrier” refers to patients having zero ApoE4 alleles.

An individual at elevated risk of Alzheimer's disease or other disease characterized by amyloid deposits of Aβ in the brain is one having one or more known risk factors (e.g., >70 years old, genetic, biochemical, family history, prodromal symptoms) placing the subject at significantly higher risk than the general population of developing the disease in a defined period, such as five years.

Mid-stage Alzheimer's disease means diagnosis of Alzheimer's disease, e.g., in accordance with DMS IV TR, and a mini-mental test score of 10-20 or equivalent score on other scales (e.g., 3-4 on Braak scale).

Late-stage Alzheimer's disease means a diagnosis of Alzheimer's disease, e.g. in accordance with DMS IV TR, and a mini-mental test score of 9 or less or equivalent score on other scales (e.g., 5-6 on Braak scale).

Alternatively, mid and late-stage Alzheimer's disease can be defined as any or all of stages 5-7 on the Global Deterioration Scale for Assessment of Primary Degenerative Dementia (GDS) (also known as the Reisberg Scale).

Statistical significance refers to p≦0.05. A change in marker relative to a baseline measurement of the marker is considered significant if the change is outside a typical margin of error in repeated measurement. For measurement of amyloid deposits by PET scanning, a typical margin of error (e.g., reproducibility of measurement on the same patient) is about 5%.

### DETAILED DESCRIPTION OF THE INVENTION

### I. General

The present invention provides methods of treating diseases characterized by amyloid deposits of Aβ with antibodies to mid- or C-terminal regions of Aβ. Although understanding of mechanisms is not required for practice of the invention, it is believed that the antibodies function at least in part by a mechanism that involves a reduction or clearing of compact or fibrillar amyloid plaques. The methods are premised in part on the result that antibodies recognizing central and/or C-terminal epitopes of Aβ can preferentially bind compact plaques relative to diffuse plaques, and thereby facilitate removal of such plaques from the brains of patients. This result is surprising in view of previous reports that mid and- terminal antibodies lack ability to bind and clear plaques, and that at least some such antibodies bind predominantly to soluble Aβ. The present data can be reconciled with the prior data in that much of the prior data was generated using plaques from the PDAPP mouse model of Alzheimer's disease, which underrepresent the proportion of compact plaques compared with an Alzheimer's patient or certain other transgenic models. Although overall binding of mid- and C-terminal antibodies to plaques in a PDAPP mouse may be regarded as low or even insignificant, mid and C-terminal antibodies bind strongly to a subset of plaques, i.e., compact plaques, present in Alzheimer's patients and certain other transgenic animal models, such as the PSAPP mouse. In consequence, mid- and C-terminal antibodies can be used for clearing plaques, and are particularly useful in patients with a relatively high proportion of dense plaques, as is often the case in mid to late stage Alzheimer's disease. Because of their preferential binding to compact plaques, mid and C-terminal antibodies are also useful in combination with N-terminal antibodies, which show more uniform recognition of different plaque types.

### II. Abeta Antibodies

The invention employs mid- or C-terminal antibodies, optionally in combination with N-terminal antibodies.

### A. C-terminal Antibodies

C-terminal antibodies of the present invention bind to an epitope within residues 29-43 of Aβ. The antibodies may or may not be end-specific. An end-specific antibody is one whose epitope includes a C-terminal amino acid with a free carboxyl group (i.e., not peptide bonded to another amino acid). End-specific amino acids preferentially bind to Aβ relative to APP or other peptide of APP spanning the C-terminus of Aβ. Antibodies can be end-specific for any of the forms of Aβ (e.g., Aβ 38, 29, 40, 41, 42, 43). C-terminal antibodies of the invention bind compact plaques relative to diffuse plaques. Some exemplary C-terminal antibodies are 2G3 antibodies (Johnson-Wood et al., PNAS 94, 1550-1555 (1997) epitope within residues Aβ₃₃₋₄₀), 14C2 antibodies (Elan Pharmaceuticals, Inc., Solomon et al., Proc Natl Acad Sci USA. 1997 Apr. 15; 94(8): 4109-4112) epitope within residues Aβ₃₃₋₄₀), 21F12 antibodies (epitope within residues Aβ₃₃₋₄₂). 21F12 is end-specific for Aβ42, 2G3 is end-specific for Aβ40 having much lower reactivity with longer forms of A. The precise epitope specificity of 14C2 has not been determined. Chimeric, humanized or veneered forms of any one of the preceding antibodies are also included, as is any antibody sharing the same six Kabat CDRs as any of 2G3, 14C2 or 21F12.

The amino acid sequences of the heavy chain and light chain variable regions of the 21F12 monoclonal antibody (Bard et al., Proc Natl Acad Sci USA. 2003 Feb. 18; 100(4): 2023-2028) are shown below (the signal sequences are italicized and underlined, and the CDRs are shown in bold with underlining):

Heavy Chain Variable Region:
(SEQ ID NO: 2)
MGWNWIFLFLLSGTAGVLSEVQLQQSGPELLKPGASVKISCKASGFTFTD
YTMHWMKQSHGKSLEWIGGINPNSGGTIYNEKFKDKATLTVDKSSRTAYM
ELRSLTSEDSAVYFCTRGVYDGYFYWGQGTLVTVSA
Light Chain Variable Region:
(SEQ ID NO: 3)
MKLPVRLLVLMFWIPASSSDVVMTQTPLSLPVSLGDQASISCRSSSLVYS
NGNTFLHWYLQKPGQSPKLLIYKVSTRFSGVPDRFSGSGSGSDFTLKISR
VEAEDLGIYFCSQTTHAPFTFGSGTKLAIR

The CDRs of the light chain variable region correspond to residues 24 to 39, residues 55 to 61, and residues 94 to 102 of SEQ ID NO:3 for CDR L1, CDR L2 and CDR L3, respectively (in which the signal sequence is numbered −19 to −1).

The CDRs of the heavy chain variable region correspond to residues 26 to 35, residues 50 to 66, and residues 99 to 106 of SEQ ID NO:2 for CDR H1, CDR H2 and CDR H3, respectively (in which the signal sequence is numbered −19 to −1).

Other C-terminal antibodies includes the 2H6 or 9TL antibodies, (Wilcock et al. (2006), supra; U.S. Pat. No. 7,807,165, and US20060057701), and the 2286 antibody (Wilcock et al. (2004), supra; WO2004032868). DNA sequences encoding the heavy and light chains of the 9TL antibody are deposited as ATCC PTA 6124 and 6125. A humanized form of the 9TL antibody is known as ponezumab. The 2286 antibody is deposited as ATCC PTA 5199. Optionally the C-terminal antibody is an antibody other than 2H6, 9TL, ponezumab or 2286. Optionally, the C-terminal antibody is an antibody not having any or all Kabat or Chothia CDRs identical to corresponding CDRs of a 2H6, 9TL or 2286 antibody. Optionally, the C-terminal antibody is an antibody not having Kabat or Chothia CDRs having at least 90% sequence identity to the CDRs of a 2H6, 9TL or 2286 antibody. Some C-terminal antibodies of the invention preferentially bind compact plaques to a greater degree than a 2H6, 9TL antibody and/or the 2286 antibody, relative to diffuse plaques.

As demonstrated in the Examples, C-terminal antibodies can be used to clear plaques, particularly compact plaques, from the brains of individuals in need of such immunotherapy (e.g., Alzheimer's disease patients).

Some antibodies of the invention bind to the same or overlapping epitope as an antibody designated 2G3, 14C2 or 21F12. Other antibodies having such a binding specificity can be produced by immunizing mice with Aβ or a portion thereof including the desired epitope, and screening resulting antibodies for binding to CD122, optionally in competition with 2G3, 14C2 or 21F12. Antibodies can also be screened against mutagenized forms of Aβ to identify an antibody showing the same or similar binding profile to collection of mutational changes as 2G3, 14C2 or 21F12. The mutations can be systematic replacement substitution with alanine (or serine if an alanine is present already) one residue at a time, or more broadly spaced intervals, throughout Aβ or through a section thereof in which an epitope is known to reside.

Antibodies having the binding specificity of a selected murine antibody (e.g., 2G3, 14C2 or 21F12) can also be produced using a variant of the phage display method. See Winter, WO 92/20791. This method is particularly suitable for producing human antibodies. In this method, either the heavy or light chain variable region of the selected murine antibody is used as a starting material. If, for example, a light chain variable region is selected as the starting material, a phage library is constructed in which members display the same light chain variable region (i.e., the murine starting material) and a different heavy chain variable region. The heavy chain variable regions can for example be obtained from a library of rearranged human heavy chain variable regions. A phage showing strong specific binding for Aβ (e.g., at least 10⁸ and preferably at least 10⁹ M⁻¹) is selected. The heavy chain variable region from this phage then serves as a starting material for constructing a further phage library. In this library, each phage displays the same heavy chain variable region (i.e., the region identified from the first display library) and a different light chain variable region. The light chain variable regions can be obtained for example from a library of rearranged human variable light chain regions. Again, phage showing strong specific binding for Aβ are selected. The resulting antibodies usually have the same or similar epitope specificity as the murine starting material.

Other antibodies can be obtained by mutagenesis of cDNA encoding the heavy and light chains of an exemplary antibody, such as 2G3, 14C2 or 21F12. Monoclonal antibodies that are at least 90%, 95% or 99% identical to 2G3, 14C2 or 21F12 in amino acid sequence of the mature heavy and/or light chain variable regions and maintain its functional properties, and/or which differ from the respective antibody by a small number of functionally inconsequential amino acid substitutions (e.g., conservative substitutions), deletions, or insertions are also included in the invention. Monoclonal antibodies having at least one and preferably all six CDR(s) as defined by Kabat that are 90%, 95%, 99% or 100% identical to corresponding CDRs of 2G3, 14C2 or 21F12 are also included.

### B. Central Epitope Antibodies

Central- or mid-epitope antibodies of the present invention recognize an epitope within residues 12-29 of Aβ, and preferentially bind compact plaques relative to diffuse plaques. Some exemplary central-epitope antibodies are the 266 antibodies (specific to an epitope within Aβ₁₆₋₂₃), the 15C11 antibody (specific to an epitope within Aβ₁₈₋₂₂), and the 22D12 antibody (Bard et al., Proc Natl Acad Sci USA. 2003 Feb. 18; 100(4): 2023-2028, specific to an epitope within Aβ₁₈₋₂₂), and chimeric, humanized or veneered forms of any one of the preceding antibodies.

A cell line producing the 266 monoclonal antibody was deposited with the American Type Culture Collection (ATCC) on Jul. 20, 2004 under the terms of the Budapest Treaty as accession number PTA-6123. Optionally, the isotype of the 266 antibody is human IgG1, IgG2 or IgG4, preferably IgG1.

The amino acid sequences of the 266 monoclonal antibody CDRs (U.S. Pat. No. 7,892,545) are as follow:

(SEQ ID NO: 4
CDR L1: Arg Ser Ser Gln Ser Leu Ile Tyr Ser Asp
Gly Asn Ala Tyr Leu His;
(SEQ ID NO: 5)
CDR L2: Lys Val Ser Asn Arg Phe Ser;
(SEQ ID NO: 6)
CDR L3: Ser Gln Ser Thr His Val Pro Trp Thr;
(SEQ ID NO: 7)
CDR H1: Arg Tyr Ser Met Ser;
(SEQ ID NO: 8)
CDR H2: Gln Ile Asn Ser Val Gly Asn Ser Thr Tyr
Tyr Pro Asp Thr Val Lys;
and
(SEQ ID NO: 9)
CDR H3: Gly Asp Tyr.

Humanized forms of the 266 antibody are described in US 20040265308, US 20040241164, WO 03/016467, and U.S. Pat. No. 7,195,761.

Light and heavy chain variable region sequences of exemplary humanized 266 antibodies are shown below (not including signal sequences):

Light Chain
Asp Xaa Val Met Thr Gln Xaa Pro Leu Ser Leu Pro
Val Xaa Xaa Gly Gln Pro Ala Ser Ile Ser Cys Arg
Ser Ser Gln Ser Leu Xaa Tyr Ser Asp Gly Asn Ala
Tyr Leu His Trp Phe Leu Gln Lys Pro Gly Gln Ser
Pro Xaa Leu Leu Ile Tyr Lys Val Ser Asn Arg Phe
Ser Gly Val Pro Asp Arg Phe Ser Gly Ser Gly Ser
Gly Thr Asp Phe Thr Leu Lys Ile Ser Arg Val Glu
Ala Glu Asp Xaa Gly Val Tyr Tyr Cys Ser Gln Ser
Thr His Val Pro Trp Thr Phe Gly Xaa Gly Thr Xaa
Xaa Glu Ile Lys Arg (SEQ ID NO: 10):

wherein: Xaa at position 2 is Val or Ile; Xaa at position 7 is Ser or Thr; Xaa at position 14 is Thr or Ser; Xaa at position 15 is Leu or Pro; Xaa at position 30 is Ile or Val; Xaa at position 50 is Arg, Gln, or Lys; Xaa at position 88 is Val or Leu; Xaa at position 105 is Gln or Gly; Xaa at position 108 is Lys or Arg; and Xaa at position 109 is Val or Leu; and

Heavy Chain
(SEQ ID NO: 11)
Xaa Val Gln Leu Val Glu Xaa Gly Gly Gly Leu Val
Gln Pro Gly Gly Ser Leu Arg Leu Ser Cys Ala Ala
Ser Gly Phe Thr Phe Ser Arg Tyr Ser Met Ser Trp
Val Arg Gln Ala Pro Gly Lys Gly Leu Xaa Leu Val
Ala Gln Ile Asn Ser Val Gly Asn Ser Thr Tyr Tyr
Pro Asp Xaa Val Lys Gly Arg Phe Thr Ile Ser Arg
Asp Asn Xaa Xaa Asn Thr Leu Tyr Leu Gln Met Asn
Ser Leu Arg Ala Xaa Asp Thr Ala Val Tyr Tyr Cys
Ala Ser Gly Asp Tyr Trp Gly Gln Gly Thr Xaa Val
Thr Val Ser Ser

wherein: Xaa at position 1 is Glu or Gln; Xaa at position 7 is Ser or Leu; Xaa at position 46 is Glu, Val, Asp, or Ser; Xaa at position 63 is Thr or Ser; Xaa at position 75 is Ala, Ser, Val or Thr; Xaa at position 76 is Lys or Arg; Xaa at position 89 is Glu or Asp; and Xaa at position 107 is Leu or Thr.

An exemplary humanized 266 antibody comprises the following light chain and heavy chain sequences (not including signal sequences):

Light Chain
(SEQ ID NO: 12)
Asp Val Val Met Thr Gln Ser Pro Leu Ser Leu Pro
Val Thr Leu Gly Gln Pro Ala Ser Ile Ser Cys Arg
Ser Ser Gln Ser Leu Ile Tyr Ser Asp Gly Asn Ala
Tyr Leu His Trp Phe Leu Gln Lys Pro Gly Gln Ser
Pro Arg Leu Leu Ile Tyr Lys Val Ser Asn Arg Phe
Ser Gly Val Pro Asp Arg Phe Ser Gly Ser Gly Ser
Gly Thr Asp Phe Thr Leu Lys Ile Ser Arg Val Glu
Ala Glu Asp Val Gly Val Tyr Tyr Cys Ser Gln Ser
Thr His Val Pro Trp Thr Phe Gly Gln Gly Thr Lys
Val Glu Ile Lys Arg Thr Val Ala Ala Pro Ser Val
Phe Ile Phe Pro Pro Ser Asp Glu Gln Leu Lys Ser
Gly Thr Ala Ser Val Val Cys Leu Leu Asn Asn Phe
Tyr Pro Arg Glu Ala Lys Val Gln Trp Lys Val Asp
Asn Ala Leu Gln Ser Gly Asn Ser Gln Glu Ser Val
Thr Glu Gln Asp Ser Lys Asp Ser Thr Tyr Ser Leu
Ser Ser Thr Leu Thr Leu Ser Lys Ala Asp Tyr Glu
Lys His Lys Val Tyr Ala Cys Glu Val Thr His Gln
Gly Leu Ser Ser Pro Val Thr Lys Ser Phe Asn Arg
Gly Glu Cys
Heavy Chain
(SEQ ID NO: 13)
Glu Val Gln Leu Val Glu Ser Gly Gly Gly Leu Val
Gln Pro Gly Gly Ser Leu Arg Leu Ser Cys Ala Ala
Ser Gly Phe Thr Phe Ser Arg Tyr Ser Met Ser Trp
Val Ary Gln Ala Pro Gly Lys Gly Leu Glu Leu Val
Ala Gln Ile Asn Ser Val Gly Asn Ser Thr Tyr Tyr
Pro Asp Thr Val Lys Gly Arg Phe Thr Ile Ser Arg
Asp Asn Ala Lys Asn Thr Leu Tyr Leu Gln Met Asn
Ser Leu Arg Ala Glu Asp Thr Ala Val Tyr Tyr Cys
Ala Ser Gly Asp Tyr Trp Gly Gln Gly Thr Leu Val
Thr Val Ser Ser Ala Ser Thr Lys Gly Pro Ser Val
Phe Pro Leu Ala Pro Ser Ser Lys Ser Thr Ser Gly
Gly Thr Ala Ala Leu Gly Cys Leu Val Lys Asp Tyr
Phe Pro Glu Pro Val Thr Val Ser Trp Asn Ser Gly
Ala Leu Thr Ser Gly Val His Thr Phe Pro Ala Val
Leu Gln Ser Ser Gly Leu Tyr Ser Leu Ser Ser Val
Val Thr Val Pro Ser Ser Ser Leu Gly Thr Gln Thr
Tyr Ile Cys Asn Val Asn His Lys Pro Ser Asn Thr
Lys Val Asp Lys Lys Val Glu Pro Lys Ser Cys Asp
Lys Thr His Thr Cys Pro Pro Cys Pro Ala Pro Glu
Leu Leu Gly Gly Pro Ser Val Phe Leu Phe Pro Pro
Lys Pro Lys Asp Thr Leu Met Ile Ser Arg Thr Pro
Glu Val Thr Cys Val Val Val Asp Val Ser His Glu
Asp Pro Glu Val Lys Phe Asn Trp Tyr Val Asp Gly
Val Glu Val His Asn Ala Lys Thr Lys Pro Arg Glu
Glu Gln Tyr Asn Ser Thr Tyr Arg Val Val Ser Val
Leu Thr Val Leu His Gln Asp Trp Leu Asn Gly Lys
Glu Tyr Lys Cys Lys Val Ser Asn Lys Ala Leu Pro
Ala Pro Ile Glu Lys Thr Ile Ser Lys Ala Lys Gly
Gln Pro Ary Glu Pro Gln Val Tyr Thr Leu Pro Pro
Ser Arg Asp Glu Leu Thr Lys Asn Gln Val Ser Leu
Thr Cys Leu Val Lys Gly Phe Tyr Pro Ser Asp Ile
Ala Val Glu Trp Glu Ser Asn Gly Gln Pro Glu Asn
Asn Tyr Lys Thr Thr Pro Pro Val Leu Asp Ser Asp
Gly Ser Phe Phe Leu Tyr Ser Lys Leu Thr Val Asp
Lys Ser Arg Trp Gln Gln Gly Asn Val Phe Ser Cys
Ser Val Met His Glu Ala Leu His Asn His Tyr Thr
Gln Lys Ser Leu Ser Leu Ser Pro Gly Lys

A cell line producing the 15C11 monoclonal antibody was deposited with the American Type Culture Collection (ATCC) on Dec. 12, 2005 under the terms of the Budapest Treaty as accession number PTA-7270. Optionally, the isotype of the 15C11 antibody is human IgG1, IgG2 or IgG4, preferably IgG1.

The amino acid sequence of the light chain variable region of the 15C11 monoclonal antibody is shown below (not including signal sequence):

(SEQ ID NO: 14)
Asp Val Val Met Thr Gln Thr Pro Leu Ser Leu Pro
Val Ser Leu Gly Asp Gln Ala Ser Ile Ser Cys Arg
Ser Ser Gln Ser Leu Val His Ser Asp Gly Asn Thr
Tyr Leu His Trp Tyr Leu Gln Lys Pro Gly Gln Ser
Pro Lys Leu Leu Ile Tyr Lys Val Ser Asn Arg Phe
Ser Gly Val Pro Asp Arg Phe Ser Gly Ser Gly Ser
Gly Thr Asp Phe Thr Leu Lys Ile Ser Arg Val Glu
Ala Glu Asp Leu Gly Val Tyr Phe Cys Ser Gln Ser
Thr His Val Trp Thr Phe Gly Gly Gly Thr Lys Leu
Glu Ile Lys

The CDRs correspond to residues 24 to 39, residues 55 to 61, and residues 94 to 101 of SEQ ID NO:14 for CDR L1, CDR L2 and CDR L3, respectively.

The amino acid sequence of the heavy chain variable region of the 15C11 monoclonal antibody is shown below:

(SEQ ID NO: 15)
Glu Val Lys Leu Val Glu Ser Gly Gly Gly Leu Val
Gln Pro Gly Gly Ser Leu Lys Leu Ser Cys Ala Ala
Ser Gly Phe Thr Phe Ser Arg Tyr Ser Met Ser Trp
Val Arg Gln Thr Pro Glu Lys Arg Leu Glu Leu Val
Ala Lys Ile Ser Asn Ser Gly Asp Asn Thr Tyr Tyr
Pro Asp Thr Leu Lys Gly Arg Phe Thr Ile Ser Arg
Asp Asn Ala Gln Asn Thr Leu Tyr Leu Gln Met Ser
Ser Leu Lys Ser Glu Asp Thr Ala Met Tyr Tyr Cys
Ala Ser Gly Asp Tyr Trp Gly Gln Gly Thr Thr Leu
Thr Val Ser Ser

The CDRs correspond to residues 26 to 35, residues 50 to 66, and residues 99 to 101 of SEQ ID NO:15 for CDR H1, CDR H2 and CDR H3, respectively.

The production of humanized forms of 15C11 is described in U.S. Pat. No. 7,625,560.

Some antibodies of the invention bind to the same or overlapping epitope as an antibody designated 266, 15C11, or 22D12. Other antibodies having such a binding specificity can be produced by immunizing mice with Aβ or a portion thereof including the desired epitope, and screening resulting antibodies for binding to Aβ, optionally in competition with 266, 15C11, or 22D12. Antibodies can also be screened against mutagenized forms of Aβ to identify an antibody showing the same or similar binding profile to collection of mutational changes as 266, 15C11, or 22D12. The mutations can be systematic replacement substitution with alanine (or serine if an alanine is present already) one residue at a time, or more broadly spaced intervals, throughout Aβ or through a section thereof in which an epitope is known to reside.

Antibodies having the binding specificity of a selected murine antibody (e.g. 266, 15C11, or 22D12) can also be produced using a variant of the phage display method. See Winter, WO 92/20791. This method is particularly suitable for producing human antibodies. In this method, either the heavy or light chain variable region of the selected murine antibody is used as a starting material. If, for example, a light chain variable region is selected as the starting material, a phage library is constructed in which members display the same light chain variable region (i.e., the murine starting material) and a different heavy chain variable region. The heavy chain variable regions can for example be obtained from a library of rearranged human heavy chain variable regions. A phage showing strong specific binding for Aβ (e.g., at least 10⁸ and preferably at least 10⁹ M⁻¹) is selected. The heavy chain variable region from this phage then serves as a starting material for constructing a further phage library. In this library, each phage displays the same heavy chain variable region (i.e., the region identified from the first display library) and a different light chain variable region. The light chain variable regions can be obtained for example from a library of rearranged human variable light chain regions. Again, phage showing strong specific binding for Aβ are selected. The resulting antibodies usually have the same or similar epitope specificity as the murine starting material.

Other antibodies can be obtained by mutagenesis of cDNA encoding the heavy and light chains of an exemplary antibody, such as 266, 15C11, or 22D12. Monoclonal antibodies that are at least 90%, 95% or 99% identical to 266, 15C11, or 22D12 in amino acid sequence of the mature heavy and/or light chain variable regions and maintain its functional properties, and/or which differ from the respective antibody by a small number of functionally inconsequential amino acid substitutions (e.g., conservative substitutions), deletions, or insertions are also included in the invention. Monoclonal antibodies having at least one and preferably all six CDR(s) as defined by Kabat that are 90%, 95%, 99% or 100% identical to corresponding CDRs of 266, 15C11, or 22D12 are also included.

As demonstrated in the Examples, central-epitope antibodies can be used to clear plaques, particularly compact plaques, from the brains of individuals in need of such immunotherapy (e.g., Alzheimer's disease patients).

### C. N-terminal Antibodies

In some methods, mid- or C-terminal antibodies are used in combination with N-terminal antibodies (i.e., antibodies binding to an epitope within residues 1-11 of Aβ and preferably within residues 1-5 or 3-7 of Aβ).

The 3D6 antibody, 10D5 antibody, and variants thereof (e g, chimeric, humanized, or veneered forms) are examples of antibodies that can be used. Both are described in US 20030165496, US 20040087777, WO 02/46237, and WO 04/080419, WO 02/088306 and WO 02/08830 and U.S. Pat. No. 7,318,923. 10D5 antibodies are also described in US 20050142131. Additional 3D6 antibodies are described in US 20060198851 and PCT/US05/45614. 3D6 is a monoclonal antibody (mAb) that specifically binds to an N-terminal epitope located in the human Aβ, specifically, residues 1-5. 10D5 is a mAb that specifically binds to an N-terminal epitope located in the human Aβ, specifically, residues 3-6. A cell line producing the 3D6 monoclonal antibody (RB96 3D6.32.2.4) was deposited with the American Type Culture Collection (ATCC) on Apr. 8, 2003 under the terms of the Budapest Treaty as accession number PTA-5130. A cell line producing the 10D5 monoclonal antibody (RB44 10D5.19.21) was deposited with the ATCC on Apr. 8, 2003 under the terms of the Budapest Treaty as accession number PTA-5129.

Bapineuzumab (international non-proprietary name designated by the World Health Organization) means a humanized 3D6 antibody comprising a light chain having a mature variable region having the amino acid sequence designated SEQ ID NO:16 and a heavy chain having a mature variable region having the amino acid sequence designated SEQ ID NO:17. (The heavy and light chain constant regions of the antibody designated bapineuzumab by WHO are human IgG1 and human kappa respectively.)

Humanized 3D6 Light Chain Variable Region
(SEQ ID NO: 16)
Asp Val Val Met Thr Gln Ser Pro Leu Ser Leu Pro
Val Thr Pro Gly Glu Pro Ala Ser Ile Ser Cys Lys
Ser Ser Gln Ser Leu Leu Asp Ser Asp Gly Lys Thr
Tyr Leu Asn Trp Leu Leu Gln Lys Pro Gly Gln Ser
Pro Gln Arg Leu Ile Tyr Leu Val Ser Lys Leu Asp
Ser Gly Val Pro Asp Arg Phe Ser Gly Ser Gly Ser
Gly Thr Asp Phe Thr Leu Lys Ile Ser Arg Val Glu
Ala Glu Asp Val Gly Val Tyr Tyr Cys Trp Gln Gly
Thr His Phe Pro Arg Thr Phe Gly Gln Gly Thr Lys
Val Glu Ile Lys
Humanized 3D6 Heavy Chain Variable Region
(SEQ ID NO: 17)
Glu Val Gln Leu Leu Glu Ser Gly Gly Gly Leu Val
Gln Pro Gly Gly Ser Leu Arg Leu Ser Cys Ala Ala
Ser Gly Phe Thr Phe Ser Asn Tyr Gly Met Ser Trp
Val Arg Gln Ala Pro Gly Lys Gly Leu Glu Trp Val
Ala Ser Ile Arg Ser Gly Gly Gly Arg Thr Tyr Tyr
Ser Asp Asn Val Lys Gly Arg Phe Thr Ile Ser Arg
Asp Asn Ser Lys Asn Thr Leu Tyr Leu Gln Met Asn
Ser Leu Arg Ala Glu Asp Thr Ala Val Tyr Tyr Cys
Val Arg Tyr Asp His Tyr Ser Gly Ser Ser Asp Tyr
Trp Gly Gln Gly Thr Leu Val Thr Val Ser Ser

A version of humanized 10D5 antibody comprising a light chain having a mature variable region having the amino acid sequence designated SEQ ID NO:18 and a heavy chain having a mature variable region having the amino acid sequence designated SEQ ID NO:19 is shown below.

Humanized 10D5 Light Chain Variable Region
(SEQ ID NO: 18)
Asp Val Leu Met Thr Gln Thr Pro Leu Ser Leu Pro
Val Ser Leu Gly Asp Gln Ala Ser Ile Ser Cys Arg
Ser Ser Gln Asn Ile Ile His Ser Asn Gly Asn Thr
Tyr Leu Glu Trp Tyr Leu Gln Lys Pro Gly Gln Ser
Pro Lys Leu Leu Ile Tyr Lys Val Ser Asn Arg Phe
Ser Gly Val Pro Asp Arg Phe Ser Gly Ser Gly Ser
Gly Thr Asp Phe Thr Leu Lys Ile Lys Lys Val Glu
Ala Glu Asp Leu Gly Ile Tyr Tyr Cys Phe Gln Gly
Ser His Val Pro Leu Thr Phe Gly Ala Gly Thr Lys
Leu Glu Leu Glu
Humanized 10D5 Heavy Chain Variable Region
(SEQ ID NO: 19)
Gln Ala Thr Leu Lys Glu Ser Gly Pro Gly Ile Leu
Gln Ser Ser Gln Thr Leu Ser Leu Thr Cys Ser Phe
Ser Gly Phe Ser Leu Ser Thr Ser Gly Met Gly Val
Ser Trp Ile Arg Gln Pro Ser Gly Lys Gly Leu Glu
Trp Leu Ala His Ile Tyr Trp Asp Asp Asp Lys Arg
Tyr Asn Pro Ser Leu Lys Ser Arg Leu Thr Ile Ser
Lys Asp Thr Ser Arg Lys Gln Val Phe Leu Lys Ile
Thr Ser Val Asp Pro Ala Asp Thr Ala Thr Tyr Tyr
Cys Val Arg Arg Pro Ile Thr Pro Val Leu Val Asp
Ala Met Asp Tyr Trp Gly Gln Gly Thr Ser Val Thr
Val Ser Ser

Another exemplary N-terminal specific antibody is 12A11 or a chimeric, humanized, veneered or nanobody form thereof. The 12A11 antibody or a variant thereof, is described in US 20050118651, US 20060198851, WO 04/108895, and WO 06/066089, all of which are incorporated by reference in their entirety herein for all purposes. 12A11 is a mAb that specifically binds to an N-terminal epitope located in the human Aβ, specifically, residues 3-7. A cell line producing the 12A11 monoclonal antibody was deposited with the American Type Culture Collection (ATCC) on Dec. 12, 2005 under the terms of the Budapest Treaty as accession number PTA-7271.

Sequences for the light and heavy chain variable regions (not including signal sequences) of an exemplary humanized 12A11 antibody are shown below.

Humanized 12A11 Light Chain Variable Region
(SEQ ID NO: 20)
Asp Val Val Met Thr Gln Ser Pro Leu Ser Leu Pro
Val Thr Pro Gly Glu Pro Ala Ser Ile Ser Cys Arg
Ser Ser Gln Ser Ile Val His Ser Asn Gly Asn Thr
Tyr Leu Glu Trp Tyr Leu Gln Lys Pro Gly Gln Ser
Pro Gln Leu Leu Ile Tyr Lys Val Ser Asn Arg Phe
Ser Gly Val Pro Asp Arg Phe Ser Gly Ser Gly Ser
Gly Thr Asp Phe Thr Leu Lys Ile Ser Arg Val Glu
Ala Glu Asp Val Gly Val Tyr Tyr Cys Phe Gln Ser
Ser His Val Pro Leu Thr Phe Gly Gln Gly Thr Lys
Leu Glu Ile Lys
Humanized 12A11 Heavy Chain Variable Region
(version 1)
(SEQ ID NO: 21)
Gln Val Gln Leu Val Glu Ser Gly Gly Gly Val Val
Gln Pro Gly Arg Ser Leu Arg Leu Ser Cys Ala Phe
Ser Gly Phe Ser Leu Ser Thr Ser Gly Met Ser Val
Gly Trp Ile Arg Gln Ala Pro Gly Lys Gly Leu Glu
Trp Leu Ala His Ile Trp Trp Asp Asp Asp Lys Tyr
Tyr Asn Pro Ser Leu Lys Ser Arg Leu Thr Ile Ser
Lys Asp Thr Ser Lys Asn Thr Val Tyr Leu Gln Met
Asn Ser Leu Arg Ala Glu Asp Thr Ala Val Tyr Tyr
Cys Ala Arg Arg Thr Thr Thr Ala Asp Tyr Phe Ala
Tyr Trp Gly Gln Gly Thr Thr Val Thr Val Ser Ser

Other exemplary N-terminal antibodies include 12B4 antibody or variants thereof (e.g., chimeric and humanized), as described in US 20040082762A1 and WO 03/077858. 12B4 is a mAb that specifically binds to an N-terminal epitope located in the human Aβ, specifically, residues 3-7.

Other exemplary N-terminal antibodies are 6C6 antibody, or a variants thereof (e.g., chimeric and humanized), as described in a US 20060165682 and WO 06/06604. 6C6 is a mAb that specifically binds to an N-terminal epitope located in the human Aβ, specifically, residues 3-7. A cell line producing the antibody 6C6 was deposited with the American Type Culture Collection (ATCC) on Nov. 1, 2005 under the terms of the Budapest Treaty as accession number PTA-7200.

Other exemplary N-terminal antibodies are 2H3 antibody, or variants thereof (e.g., chimeric or humanized), as described in US 20060257396. 2H3 is a mAb that specifically binds to an N-terminal epitope located in the human Aβ, specifically, residues 2-7. A cell line producing the antibody 2H3 was deposited with the American Type Culture Collection on Dec. 13, 2005 under the terms of the Budapest Treaty as accession number PTA-7267.

Other exemplary N-terminal antibodies include 3A3 and variants thereof (e.g., chimeric or humanized), as described in US 20060257396. 3A3 is a mAb that specifically binds to an N-terminal epitope located in the human Aβ, specifically, residues 3-7. A cell line producing the antibody 3A3 was deposited with the American Type Culture Collection (ATCC) on Dec. 13, 2005 under the terms of the Budapest Treaty as accession number PTA-7269.

### D. General Characteristics

The following characteristics apply to any of the C-terminal, mid or N-terminal antibodies just described. Antibodies can be polyclonal or monoclonal. Polyclonal sera typically contain mixed populations of antibodies specifically binding to several epitopes along the length of APP. However, polyclonal sera can be specific to a particular segment of Aβ such as Aβ1-11) without specifically binding to other segments of Aβ. Preferred antibodies are chimeric, humanized or veneered (see Queen et al., Proc. Natl. Acad. Sci. USA 86:10029-10033 (1989) and WO 90/07861, U.S. Pat. No. 5,693,762, U.S. Pat. No. 5,693,761, U.S. Pat. No. 5,585,089, U.S. Pat. No. 5,530,101 and Winter, U.S. Pat. No. 5,225,539), or human (Lonberg et al., WO 93/12227 (1993); U.S. Pat. No. 5,877,397, U.S. Pat. No. 5,874,299, U.S. Pat. No. 5,814,318, U.S. Pat. No. 5,789,650, U.S. Pat. No. 5,770,429, U.S. Pat. No. 5,661,016, U.S. Pat. No. 5,633,425, U.S. Pat. No. 5,625,126, U.S. Pat. No. 5,569,825, U.S. Pat. No. 5,545,806, Nature 148, 1547-1553 (1994), Nature Biotechnology 14, 826 (1996), Kucherlapati, WO 91/10741 (1991)) EP1481008, Bleck, Bioprocessing Journal 1 (September/October 2005), US 2004132066, US 2005008625, WO 04/072266, WO 05/065348, WO 05/069970, and WO 06/055778.

The production of other non-human monoclonal antibodies, e.g., murine, guinea pig, primate, rabbit or rat, against Aβ can be accomplished by, for example, immunizing the animal with Aβ or a fragment thereof. See Harlow & Lane, Antibodies, A Laboratory Manual (CSHP NY, 1988) (incorporated by reference for all purposes). Optionally, the immunogen can be administered with an adjuvant. Several types of adjuvant can be used as described below. Complete Freund's adjuvant followed by incomplete adjuvant is preferred for immunization of laboratory animals. Rabbits or guinea pigs are typically used for making polyclonal antibodies. Mice are typically used for making monoclonal antibodies. Antibodies are screened for specific binding to a desired epitope within Aβ.

A humanized antibody is a genetically engineered antibody in which the CDRs from a non-human “donor” antibody are grafted into human “acceptor: antibody sequences (see, e.g., Queen, U.S. Pat. Nos. 5,530,101 and 5,585,089; Winter, U.S. Pat. No. 5,225,539, Carter, U.S. Pat. No. 6,407,213, Adair, U.S. Pat. Nos. 5,859,205 6,881,557, Foote, U.S. Pat. No. 6,881,557). The acceptor antibody sequences can be, for example, a mature human antibody sequence, a consensus sequence of human antibody sequences, or a germline region sequence. Thus, a humanized antibody is an antibody having some or all CDRs entirely or substantially from a donor antibody and variable region framework sequences and constant regions entirely or substantially from human antibody sequences. Similarly a humanized heavy chain has at least one, two and usually all three CDRs entirely or substantially from a donor antibody heavy chain, and a heavy chain variable region framework sequence and heavy chain constant region, if present, substantially from human heavy chain variable region framework and constant region sequences. Similarly a humanized light chain has at least one, two and usually all three CDRs entirely or substantially from a donor antibody light chain, and a light chain variable region framework sequence and light chain constant region, if present, substantially from human light chain variable region framework and constant region sequences. Other than nanobodies and dAbs, a humanized antibody comprises a humanized heavy chain and a humanized light chain. A CDR in a humanized antibody is substantially from a corresponding CDR in a non-human antibody when at least 85%, 90%, 95% or 100% of corresponding residues (as defined by Kabat) are identical between the respective CDRs. The variable region framework sequences of an antibody chain or the constant region of an antibody chain are substantially from a human variable region framework sequence or human constant region respectively when at least 85, 90, 95 or 100% of corresponding residues defined by Kabat are identical.

Although humanized antibodies often incorporate all six CDRs (preferably as defined by Kabat) from a mouse antibody, they can also be made with less than all CDRs (e.g., at least 3, 4, 5) CDRs from a mouse antibody (e.g., Pascalis et al., J. Immunol. 169:3076, 2002; Vajdos et al., Journal of Molecular Biology, 320: 415-428, 2002; Iwahashi et al., Mol. Immunol. 36:1079-1091, 1999; Tamura et al, Journal of Immunology, 164:1432-1441, 2000).

In some antibodies only part of the CDRs, namely the subset of CDR residues required for binding, termed the SDRs, are need to retain binding in a humanized antibody. CDR residues not contacting antigen and not in the SDRs can be identified based on previous studies (for example residues H60-H65 in CDR H2 are often not required), from regions of Kabat CDRs lying outside Chothia hypervariable loops (Chothia, J. Mol. Biol. 196:901, 1987), by molecular modeling and/or empirically, or as described in Gonzales et al., Mol. Immunol. 41: 863, 2004. In such humanized antibodies at positions in which one or more donor CDR residues is absent or in which an entire donor CDR is omitted, the amino acid occupying the position can be an amino acid occupying the corresponding position (by Kabat numbering) in the acceptor antibody sequence. The number of such substitutions of acceptor for donor amino acids in the CDRs to include reflects a balance of competing considerations. Such substitutions are potentially advantageous in decreasing the number of mouse amino acids in a humanized antibody and consequently decreasing potential immunogenicity. However, substitutions can also cause changes of affinity, and significant reductions in affinity are preferably avoided. Positions for substitution within CDRs and amino acids to substitute can also be selected empirically.

The human acceptor antibody sequences can optionally be selected from among the many known human antibody sequences to provide a high degree of sequence identity (e.g., 65-85% identity) between a human acceptor sequence variable region frameworks and corresponding variable region frameworks of a donor antibody chain.

Certain amino acids from the human variable region framework residues can be selected for substitution based on their possible influence on CDR conformation and/or binding to antigen. Investigation of such possible influences is by modeling, examination of the characteristics of the amino acids at particular locations, or empirical observation of the effects of substitution or mutagenesis of particular amino acids.

For example, when an amino acid differs between a murine variable region framework residue and a selected human variable region framework residue, the human framework amino acid can be substituted by the equivalent framework amino acid from the mouse antibody when it is reasonably expected that the amino acid:
(1) noncovalently binds antigen directly,
(2) is adjacent to a CDR region,
(3) otherwise interacts with a CDR region (e.g. is within about 6 A of a CDR region).

Other candidates for substitution are acceptor human framework amino acids that are unusual for a human immunoglobulin at that position. These amino acids can be substituted with amino acids from the equivalent position of the mouse donor antibody or from the equivalent positions of more typical human immunoglobulins. Other candidates for substitution are acceptor human framework amino acids that are unusual for a human immunoglobulin at that position.

A chimeric antibody is an antibody in which the mature variable regions of light and heavy chains of a non-human antibody (e.g., a mouse) are combined with human light and heavy chain constant regions. Such antibodies substantially or entirely retain the binding specificity of the mouse antibody, and are about two-thirds human sequence.

A veneered antibody includes at least one veneered antibody chain (i.e., at least one veneered light or heavy chain and usually both). A veneered antibody chain is n antibody chain (i.e., a light or heavy chain, respectively) having (i) a variable region that includes complementarity determining regions (CDRs) (e.g., at least one CDR, preferably two CDRs, more preferably three CDRs) substantially from a non-human antibody (e.g., rodent, and optionally, mouse) and a variable region framework substantially from a non-human antibody (e.g., mouse), except that surface exposed residues of the variable region framework (preferably all such residues) are substantially from a human antibody sequence (e.g., a consensus sequence), and (ii) constant regions entirely or substantially from a human antibody constant region. CDRs are typically as defined by Kabat, but alternatively can be as defined by Chothia or a composite of the CDR regions defined by Kabat and Chothia. Human antibodies against Aβ are provided by a variety of techniques described below. Some human antibodies are selected by competitive binding experiments, by the phage display method of Winter, above, or otherwise, to have the same epitope specificity as a particular mouse antibody, such as one of the mouse monoclonals described in the examples. Human antibodies can also be screened for a particular epitope specificity by using only a fragment of Aβ as the immunogen, and/or by screening antibodies against a collection of deletion mutants of Aβ.

Methods for producing human antibodies include the trioma method of Oestberg et al., Hybridoma 2:361-367 (1983); Oestberg, U.S. Pat. No. 4,634,664; and Engleman et al., U.S. Pat. No. 4,634,666, use of transgenic mice including human immunoglobulin genes (see, e.g., Lonberg et al., WO93/12227 (1993); U.S. Pat. No. 5,877,397, U.S. Pat. No. 5,874,299, U.S. Pat. No. 5,814,318, U.S. Pat. No. 5,789,650, U.S. Pat. No. 5,770,429, U.S. Pat. No. 5,661,016, U.S. Pat. No. 5,633,425, U.S. Pat. No. 5,625,126, U.S. Pat. No. 5,569,825, U.S. Pat. No. 5,545,806, Nature 148, 1547-1553 (1994), Nature Biotechnology 14, 826 (1996), Kucherlapati, WO 91/10741 (1991) and phage display methods (see, e.g., Dower et al., WO 91/17271 and McCafferty et al., WO 92/01047, U.S. Pat. No. 5,877,218, U.S. Pat. No. 5,871,907, U.S. Pat. No. 5,858,657, U.S. Pat. No. 5,837,242, U.S. Pat. No. 5,733,743 and U.S. Pat. No. 5,565,332.

Any of the antibodies or antibody fragments described herein can be designed or prepared using standard methods, as disclosed, e.g., in US 20040038304, US 20070020685, US 200601660184, US 20060134098, US 20050255552, US 20050130266, US 2004025363, US 20040038317, US 20030157579, and U.S. Pat. No. 7,335,478.

Any of the antibodies or antibody fragments can be subject to treatments that add or remove posttranslationional modifications, such as phosphorylation, carboxylation or glyocylation. For example, glycosylation can be removed by treatment with a glycosidase, such as N-glycosidase F, or by mutagenesis of residues subject to glycosylation.

Any of the antibodies described above can be produced with different isotypes or mutant isotypes to control the extent of binding to different Fcγ receptors. Antibodies lacking an Fc region (e.g., Fab fragments) lack binding to Fcγ receptors. Selection of isotype affects binding to Fcγ receptors. Human, chimeric or humanized antibodies incorporate constant regions that are substantially or entirely human. The most common isotypes are human IgG1, IgG2, IgG3 and IgG4. Thus humanized, chimeric or veneered forms of any of 2G3, 14C2, 21F12, 266, 15C11 and 22D12 can have any of human IgG1, IgG2, IgG3 and IgG4 isotypes. The respective affinities of various human IgG isotypes for the three Fcγ receptors, FcγRI, FcγRII, and FcγRIII, have been determined. (See Ravetch & Kinet, Annu. Rev. Immunol. 9, 457 (1991)). FcγRI is a high affinity receptor that binds to IgGs in monomeric form, and the latter two are low affinity receptors that bind IgGs only in multimeric form. In general, both IgG1 and IgG3 have significant binding activity to all three receptors, IgG4 to FcγRI, and IgG2 to only one type of FcγRII called IIa_{LR} (see Parren et al., J. Immunol. 148, 695 (1992). Human IgG1 and IgG3 support complement function whereas human IgG2 and IgG4 do not. Human isotype IgG1 is usually selected when effector functions are desired and human IgG2 or IgG4 when they are not. Human IgG1 is preferred in the present methods.

Mutations on, adjacent, or close to sites in the hinge link region (e.g., replacing residues 234, 235, 236 and/or 237 with another residue) in all of the isotypes reduce affinity for Fcγ receptors, particularly FcγRI receptor (see, e.g., U.S. Pat. No. 6,624,821). Optionally, positions 234, 236 and/or 237 are substituted with alanine and position 235 with glutamine. (See, e.g., U.S. Pat. No. 5,624,821.) Position 236 is missing in the human IgG2 isotype. Exemplary segments of amino acids for positions 234, 235 and 237 for human IgG2 are Ala Ala Gly, Val Ala Ala, Ala Ala Ala, Val Glu Ala, and Ala Glu Ala. A preferred combination of mutants is L234A, L235A, and G237A for human isotype IgG1. A particular preferred N-terminal antibody is bapineuzumab having human isotype IgG and these three mutations of the Fc region of human IgG1. Other substitutions that decrease binding to Fcγ receptors are an E233P mutation (particularly in mouse IgG1) and D265A (particularly in mouse IgG2a). Other examples of mutations and combinations of mutations reducing Fc and/or Clq binding include (E318A/K320A/R322A (particularly in mouse IgG1), and L235A/E318A/K320A/K322A (particularly in mouse IgG2a). Similarly, residue 241 (Ser) in human IgG4 can be replaced, e.g., with proline to disrupt Fc binding.

Additional mutations can be made to the constant region to modulate effector activity. For example, mutations can be made to the IgG2a constant region at A330S, P331S, or both. For IgG4, mutations can be made at E233P, F234V and L235A, with G236 deleted, or any combination thereof. IgG4 can also have one or both of the following mutations S228P and L235E. The use of disrupted constant region sequences to modulate effector function is further described, e.g., in WO 06/118,959 and WO 06/036291.

Additional mutations can be made to the constant region of human IgG to modulate effector activity (see, e.g., WO 06/03291). These include the following substitutions: (i) A327G, A330S, P331S; (ii) E233P, L234V, L235A, G236 deleted; (iii) E233P, L234V, L235A; (iv) E233P, L234V, L235A, G236 deleted, A327G, A330S, P331S; and (v) E233P, L234V, L235A, A327G, A330S, P331S to human IgG1.

The affinity of an antibody for the FcR can be altered by mutating certain residues of the heavy chain constant region. For example, disruption of the glycosylation site of human IgG1 can reduce FcR binding, and thus effector function, of the antibody (see, e.g., WO 06/036291). The tripeptide sequences NXS, NXT, and NXC, where X is any amino acid other than proline, are the enzymatic recognition sites for glycosylation of the N residue. Disruption of any of the tripeptide amino acids, particularly in the CH2 region of IgG, will prevent glycosylation at that site. For example, mutation of N297 of human IgG1 prevents glycosylation and reduces FcR binding to the antibody.

Human constant regions show allotypic variation and isoallotypic variation between different individuals, that is, the constant regions can differ in different individuals at one or more polymorphic positions. Isoallotypes differ from allotypes in that sera recognizing an isoallotype binds to a non-polymorphic region of one or more other isotypes. A preferred allotype of the IgG1 constant region is Glmz which has Glu at position 356 and Met at position 358. A preferred allotype of the kappa constant region is Km3, which has an Ala at position 153 and a Val at position 191. A different allotype Km(1) has Val and Leu at positions 153 and 191 respectively. Allotypic variants are reviewed by J Immunogen 3: 357-362 (1976) and Loghem, Monogr Allergy 19: 40-51 (1986). Other allotypic and isoallotypic variants of the constant regions are included. Also included are constant regions having any permutation of residues occupying polymorphic positions in natural allotypes. Examples of other heavy chain IgG1 allotypes include: Glm(f), Glm(a) and Glm(x). Glm(f) differs from Glm(z) in that it has an Arg instead of a Lys at position 214. Glm(a) has amino acids Arg, Asp, Glu, Leu at positions 355-358.

### III. Active Immunization

Antibodies to C-terminal, mid or N-terminal epitopes can also be generated in situ in a patient by active immunization with immunogenic fragments of Aβ or analogs thereof that induce such antibodies. Preferred fragments have about 5-12, 5-10, and more preferably 6-9 contiguous residues of Aβ. For generating antibodies to central epitopes, the 5-12, 5-10 or more preferably 6-9 contiguous residues are within residues 12 and 28 of Aβ. For generating antibodies to C-terminal epitopes the 5-12, 5-10 or more preferably 6-9 contiguous residues are within residues 29-43 of Aβ. For generating antibodies to N-terminal epitopes the 5-12 or 6-9 contiguous residues are within residues 1-11 of Aβ.

Preferred fragments for inducing antibodies to central epitopes of Aβ include Aβ15-21, Aβ16-22, Aβ17-23, Aβ18-24, Aβ19-25, Aβ15-22, Aβ16-23, Aβ17-24, Aβ18-25, Aβ15-23, Aβ16-24, Aβ17-25, Aβ18-26, Aβ15-24, Aβ16-25, and Aβ15-25. Aβ16-23 is particularly preferred meaning a fragment including residues 16-23 of Aβ and lacking other residues of Aβ. At least some of the antibodies induced by such fragments preferentially bind to compact plaques over diffuse plaques as is the case for exemplified central region monoclonal antibodies, such as 266.

Preferred fragments for inducing antibodies to C-terminal epitopes include C-terminal fragments of Aβ39, 40, 41, 42 or 43 of 5-12 and preferably 6-9 contiguous amino acids between residues 29 and 43 of Aβ. As least some of the antibodies induced by such fragments preferentially bind to compact plaques over diffuse plaques as is the case for exemplified C-terminal antibodies.

Preferred fragment for inducing N-terminal antibodies include fragments beginning at a residue between 1-3 of Aβ and ending at a residue between 7-11 of Aβ. Exemplary N-terminal fragments include Aβ1-5, 1-6, 1-7, 1-10, and 3-7 with 1-7 being particularly preferred.

Fragments preferably lack T-cell epitopes that would induce T-cells against Aβ. Generally, T-cell epitopes are greater than 10 contiguous amino acids. Therefore, preferred fragments of Aβ are of size 5-10 or preferably 6-9 contiguous amino acids; i.e., sufficient length to generate an antibody response without generating a T-cell response. Absence of T-cell epitopes is preferred because these epitopes are not needed for immunogenic activity of fragments, and may cause an undesired inflammatory response in a subset of patients.

Fragments are usually fragments of natural Aβ (e.g., Aβ 1-42 shown in SEQ ID NO:1) but can include unnatural amino acids or modifications of N or C terminal amino acids at a one, two, five, ten or even all positions. For example, the natural aspartic acid residue at position 1 and/or 7 of Aβ can be replaced with iso-aspartic acid. Examples of unnatural amino acids are D, alpha, alpha-disubstituted amino acids, N-alkyl amino acids, lactic acid, 4-hydroxyproline, γ-carboxyglutamate, epsilon-N,N,N-trimethyllysine, epsilon-N-acetyllysine, 0-phosphoserine, N-acetylserine, N-formylmethionine, 3-methylhistidine, 5-hydroxylysine, omega-N-methylarginine, β-alanine, ornithine, norleucine, norvaline, hydroxproline, thyroxine, γ-amino butyric acid, homoserine, citrulline, and isoaspartic acid. Some fragments are all-D peptides, e.g., all-D Aβ or all-D Aβ fragment, and all-D peptide analogs. Fragments can be screened for prophylactic or therapeutic efficacy in transgenic animal models in comparison with untreated or placebo controls.

Fragments are typically conjugated to carrier molecules, typically a carrier polypeptide, which provides a T-cell epitope, and thus help elicit an immune response against the fragment conjugated to the carrier. A single agent can be linked to a single carrier, multiple copies of an agent can be linked to multiple copies of a carrier, which are in turn linked to each other, multiple copies of an agent can be linked to a single copy of a carrier, or a single copy of an agent can be linked to multiple copies of a carrier, or different carriers. Suitable carriers include serum albumins, keyhole limpet hemocyanin, immunoglobulin molecules, thyroglobulin, ovalbumin, tetanus toxoid, or a toxoid from other pathogenic bacteria, such as diphtheria (e.g., CRM₁₉₇), E. coli, cholera, or H. pylori, or an attenuated toxin derivative. T cell epitopes are also suitable carrier molecules. Some conjugates can be formed by linking agents of the invention to an immunostimulatory polymer molecule (e.g., tripalmitoyl-S-glycerine cysteine (Pam₃Cys), mannan (a mannose polymer), or glucan (a β 1→2 polymer)), cytokines (e.g., IL-1, IL-1 alpha and β peptides, IL-2, γ-INF, IL-10, GM-CSF), and chemokines (e.g., MIP1-α and β, and RANTES) Immunogenic agents can also be linked to peptides that enhance transport across tissues, as described in O'Mahony, WO 97/17613 and WO 97/17614 Immunogens may be linked to the carries with or with out spacers amino acids (e.g., gly-gly).

Additional carriers include virus-like particles. Virus-like particles (VLPs), also called pseudovirions or virus-derived particles, represent subunit structures composed of multiple copies of a viral capsid and/or envelope protein capable of self assembly into VLPs of defined spherical symmetry in vivo. (Powilleit, et al., (2007) PLoS ONE 2(5):e415.) These particles have been found to be useful as antigen delivery systems. VLPs can be produced and readily purified in large quantities and due to their particulate nature and high molecular weights. VLPs induce an immune response without additional application of an adjuvant. (Ulrich et al., (1996) Intervirology 39:126-132.) Exemplary chimeric particles useful as VLP antigen delivery systems include those based on hepatitis B virus, human immunodeficiency virus (HIV), yeast retrotransposon Ty, yeast totivirus L-A, parvovirus, influenza virus, Norwalk virus, rotavirus, adeno-associated virus, bluetongue virus, hepatitis A virus, human papillomavirus, measles virus, polyoma virus and RNA phage virus, as well as those based on various retroviruses and lentiviruses. For review, see Lechner, et al. (2002) Intervirology 45:212-217.

The core protein of hepatitis B virus (HBcAg) is a common VLP used for carrying foreign antigens (see Koletzki et al., (1997) J Gen Vir 78:2049-2053). Briefly, HBcAg can be used as a core to construct VLPs that present extended foreign protein segments. The method employs a construct having a linker sequence between the a C-terminally truncated HBcAg and a foreign protein sequence that contains a stop codon. Truncated HBcAg/foreign protein chimera is expressed utilizing a read through mechanism based on the opal TGA-Trp mutation for expression in an E. coli suppressor strain. The method described by Koletzki et al. allows for incorporation of long foreign protein sequences into VLPs, allowing for a greater variety of antigens to be carried by the VLP.

The HIV virus Gag protein can be used as an antigen carrier system (see Griffiths et al., (1993) J Virol. 67(6):3191-3198). Griffiths utilized the V3 loop of HIV, which is the principle neutralizing determinant of the HIV envelope. The Gag:V3 fusion proteins assembled in vivo into hybrid Gag particles, designated virus-derived particles (VDPs). The VDPs induce both humoral and cellular responses. As the V3 loop contains a CTL epitope, immunization with Gag:V3 induces a CTL response to the V3 protein portion of the VLP.

A hybrid HIV: Ty VLP can also be used (see Adams et al., (1987) Nature 329(3):68-70). The HIV:Ty VLP employs the p1 protein of the yeast transposon Ty. The first 381 amino acids of p1 are sufficient for VLP formation. The HIV:Ty fusion proteins are capable of assembling into VLPs in vivo, as well as inducing an immune response to the HIV antigen carried by the VLP. VLPs using the Ty p1 protein can also contain p1 fused to the whole of an alpha2-interferon, the product of the bovine papilloma virus E1 and E2 genes, and a portion of an influenza hemagglutinin. Each of these Ty fusions formed VLPs and were capable of inducing production of antisera to the non-Ty VLP component.

VLPs can also be designed from variants of the yeast totivirus L-A (see Powilleit et al. (2007) PLOS One 2(5):e415). The Pol gene of the L-A virus can be replaced with an appropriate antigen to induce a specific immune response, demonstrating that yeast VLPs are effective antigen carriers.

Recombinant, nonreplicative parvovirus-like particles can also be used as antigen carriers. (Sedlik, et al. (1997) PNAS 94:7503-7508.) These particles allow the carried antigens into the cytosol so they enter the class I-restricted immunological pathway, thus stimulating cytotoxic T-lymphocyte (CTL) mediated responses. Sedlik specifically used PPV:VLP, which contained the VP2 capsid protein of the parvovirus and residues 118-132 from the lymphocytic choriomeningitis virus (LCMV) was inserted into the VP2 capsid protein. The PPV:VLP containing LCMV was capable of inducing an immune response to LCMV and elicited immunological protection against lethal viral doses in pre-immunized mice.

VLPs can also comprise replication incompetent influenza that lack the influenza NS2 gene, the gene essential for viral replication. (Watanabe, et al. (1996) J Virol. 76(2):767-773.) These VLPs infect mammalian cells and allow expression of foreign proteins.

Norwalk virus (NV)-based VLPs can also be used as vehicles for immunogen delivery. (Ball, et al. (1999) Gastroenterology 117:40-48.) The NV genome has three open reading frames (ORFs 1-3). Recombinant baculovirus expression of ORFs 2 and 3 allows for spontaneous assembly of high yields of recombinant Norwalk virus (rNV) VLPs.

Fragments are often administered with pharmaceutically acceptable adjuvants. The adjuvant increases the titer of induced antibodies and/or the binding affinity of induced antibodies relative to the situation if the peptide were used alone. A variety of adjuvants can be used in combination with an immunogenic fragment of Aβ, to elicit an immune response. Preferred adjuvants augment the intrinsic response to an immunogen without causing conformational changes in the immunogen that affect the qualitative form of the response. Preferred adjuvants include aluminum hydroxide and aluminum phosphate, 3 De-O-acylated monophosphoryl lipid A (MPL™) (see GB 2220211 (RIBI ImmunoChem Research Inc., Hamilton, Mont., now part of Corixa). Stimulon™ QS-21 is a triterpene glycoside or saponin isolated from the bark of the Quillaja Saponaria Molina tree found in South America (see Kensil et al., in Vaccine Design: The Subunit and Adjuvant Approach (eds. Powell & Newman, Plenum Press, NY, 1995); U.S. Pat. No. 5,057,540), (Aquila BioPharmaceuticals, Framingham, Mass.; now Antigenics, Inc., New York, N.Y.). Other adjuvants are oil in water emulsions (such as squalene or peanut oil), optionally in combination with immune stimulants, such as monophosphoryl lipid A (see Stoute et al., N. Engl. J. Med. 336, 86-91 (1997)), pluronic polymers, and killed mycobacteria. Another adjuvant is CpG (WO 98/40100). Adjuvants can be administered as a component of a therapeutic composition with an active agent or can be administered separately, before, concurrently with, or after administration of the therapeutic agent.

A preferred class of adjuvants is aluminum salts (alum), such as alum hydroxide, alum phosphate, alum sulfate. Such adjuvants can be used with or without other specific immunostimulating agents such as MPL or 3-DMP, QS-21, polymeric or monomeric amino acids such as polyglutamic acid or polylysine. Another class of adjuvants is oil-in-water emulsion formulations. Such adjuvants can be used with or without other specific immunostimulating agents such as muramyl peptides (e.g., N-acetylmuramyl-L-threonyl-D-isoglutamine (thr-MDP), N-acetyl-normuramyl-L-alanyl-D-isoglutamine (nor-MDP), N-acetylmuramyl-L-alanyl-D-isoglutaminyl-L-alanine-2-(1′-2′dipalmitoyl-sn-glycero-3-hydroxyphosphoryloxy)-ethylamine (MTP-PE), N-acetylglucsaminyl-N-acetylmuramyl-L-A1-D-isoglu-L-Ala-dipalmitoxy propylamide (DTP-DPP) Theramide™), or other bacterial cell wall components. Oil-in-water emulsions include (a) MF59 (WO 90/14837), containing 5% Squalene, 0.5% Tween 80, and 0.5% Span 85 (optionally containing various amounts of MTP-PE) formulated into submicron particles using a microfluidizer such as Model 110Y microfluidizer (Microfluidics, Newton Mass.), (b) SAF, containing 10% Squalene, 0.4% Tween 80, 5% pluronic-blocked polymer L121, and thr-MDP, either microfluidized into a submicron emulsion or vortexed to generate a larger particle size emulsion, and (c) Ribi™ adjuvant system (RAS), (Ribi ImmunoChem, Hamilton, Mont.) containing 2% squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detox™).

Another class of preferred adjuvants is saponin adjuvants, such as Stimulon™ (QS-21, Aquila, Framingham, Mass.) or particles generated therefrom such as ISCOMs (immunostimulating complexes) and ISCOMATRIX. Other adjuvants include RC-529, GM-CSF and Complete Freund's Adjuvant (CFA) and Incomplete Freund's Adjuvant (IFA). Other adjuvants include cytokines, such as interleukins (e.g., IL-1 α and β peptides, IL-2, IL-4, IL-6, IL-12, IL13, and IL-15), macrophage colony stimulating factor (M-CSF), granulocyte-macrophage colony stimulating factor (GM-CSF), tumor necrosis factor (TNF), chemokines, such as MIP1α and β and RANTES. Another class of adjuvants is glycolipid analogues including N-glycosylamides, N-glycosylureas and N-glycosylcarbamates, each of which is substituted in the sugar residue by an amino acid, as immuno-modulators or adjuvants (see U.S. Pat. No. 4,855,283). Heat shock proteins, e.g., HSP70 and HSP90, may also be used as adjuvants.

An adjuvant can be administered with an immunogen as a single composition, or can be administered before, concurrent with or after administration of the immunogen. Immunogen and adjuvant can be packaged and supplied in the same vial or can be packaged in separate vials and mixed before use Immunogen and adjuvant are typically packaged with a label indicating the intended therapeutic application. If immunogen and adjuvant are packaged separately, the packaging typically includes instructions for mixing before use. The choice of an adjuvant and/or carrier depends on the stability of the immunogenic formulation containing the adjuvant, the route of administration, the dosing schedule, the efficacy of the adjuvant for the species being vaccinated, and, in humans, a pharmaceutically acceptable adjuvant is one that has been approved or is approvable for human administration by pertinent regulatory bodies. For example, Complete Freund's adjuvant is not suitable for human administration. Alum, MPL and QS-21 are preferred. Optionally, two or more different adjuvants can be used simultaneously. Preferred combinations include alum with MPL, alum with QS-21, MPL with QS-21, MPL or RC-529 with GM-CSF, and alum, QS-21 and MPL together. Also, Incomplete Freund's adjuvant can be used (Chang et al., Advanced Drug Delivery Reviews 32, 173-186 (1998)), optionally in combination with any of alum, QS-21, and MPL and all combinations thereof.

### IV. Patients Amenable to Treatment

C-terminal and central-epitope antibodies or fragments of Aβ that induce such antibodies can be used in an effective regime to treat diseases and/or conditions associated with compact plaques of Aβ (e.g., Alzheimer's disease, Down's syndrome and some forms of Parkinson's disease). The proportion of compact plaques, also referred to as “fibrillar plaques” to total plaques in preclinical AD is 22%, but rises to 49% in end-stage AD. Dickson and Vickers, Neuroscience 105:99-107 (2001). The majority of these compact plaques (82%) are associated with dystrophic neuritis. Id. Thus, progression of AD dementia is associated with a shift to a higher proportion of compact plaques that induce local neuritic dystrophy.

Because the representation of compact plaques increases with disease progression, patients suffering from mid- to late-stage Alzheimer's disease tend to have a relatively high representation of compact plaques. Thus, patients having mid to late stage Alzheimer's disease diagnosed e.g., from a cognitive scale can be treated with a central or C-terminal antibody or a fragment of Aβ that induces such an antibody with or without an individualized assessment of representation of compact plaques.

Alternatively, patients can be assessed for treatment with a central or C-terminal antibody or fragment of Aβ that induces such an antibody from their representation of compact plaques without necessarily otherwise assessing their stage of disease progression, such as on a cognitive scale. The representation of compact plaques can be determined by PET scanning as discussed in more detail below. For calculating a ratio the number of compact plaques is compared with the total number of plaques (i.e., compact plaques, diffuse plaques and dense core plaques). Dense core plaques are included in total plaques but are not scored as compact plaques. Plaques (of all types) are measured within defined cross-section(s) or a volume(s) or region(s) of the brain. The proportion of compact plaques signaling initiation of treatment can be at least 25% of total plaques. In some methods, the proportion of compact plaques to total plaques is at least 30%, at least 35%, at least 40%, at least 45%, or at least 50% of total plaques before initiating treatment with a central or C-terminal antibody. In some patients, the proportion of compact plaques to total plaques is, or is at least, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% before initiating treatment with a central or C-terminal antibody.

C-terminal and central-epitope antibodies have a different binding profile to compact and diffuse plaques than N-terminal antibodies (i.e., C-terminal and central epitope antibodies preferentially bind to compact plaques). Also many amyloid plaques in AD brains are N-terminally truncated and modified, starting with pyroglumate at position 3. Harigaya et al., Biochem Biophys Res Commun 276:422-427 (2000); Guntert et al., Neuroscience 143:461-475 (2006). Thus, antibodies that recognize C-terminal or central epitopes, recognize truncated molecules of Aβ not recognized by N-terminal epitope antibodies. Because of the different binding specificities, treatment with a C-terminal or central epitope antibody or a fragment of Aβ that induces such an antibody can in some methods be combined with treatment with an N-terminal antibody, or a fragment of Aβ that induces such an antibody. The combination of antibodies can result in removal of more amyloid deposits than use of individual antibodies. The different antibodies can be administered sequentially or concurrently.

In some methods, patients are first treated with an N-terminal epitope antibody or fragment that induces such an antibody. If treatment with the N-terminal epitope antibody results in lack of clearance, suboptimal or incomplete clearance of compact plaques, such patients can then be treated with a C-terminal or central epitope antibody. Lack of clearance, suboptimal or incomplete clearance can be determined by PET imaging as discussed below or can be determined inferentially from other biomarkers or inadequate inhibition of cognitive decline. Cognitive measures include ADAS-CO11, ADAS-CO12, DAASD, CDR-SB, NTB, NPI, MMSE) and biomarkers include [18F]FDG, MRI markers (BBSI and VBSI), and CSF markers Aβ≧42, tau and phospho-tau. However, changes detected by PET imaging often precede changes in biomarkers or cognitive tests.

In some methods, patients are first treated with a C-terminal or central-epitope antibody, or fragment of Aβ that induces such an antibody. If treatment results in lack of clearance, incomplete or suboptimal clearance of diffuse plaques, then an N-terminal epitope antibody can be administered. Lack of clearance, incomplete or suboptimal clearance of diffuse plaques can be determined by PET imaging or can be inferred from biomarkers or from inadequate inhibition of cognitive decline.

In other methods, an N-terminal antibody or fragment of Aβ that induces such an antibody and a C-terminal or central-epitope antibody or a fragment of Aβ that induces such an antibody are administered concurrently. Concurrent administration includes administering the antibodies at the same time (e.g., as a mixed formulation) or separately but in overlapping regimes so that therapeutic concentrations of both antibodies exist in the serum for an extended period of time (e.g., at least 1, 3, 6 or 12 months). In such methods, the different specificities of the antibodies can combine to reduce or at least inhibit further increase of both compact and diffuse plaques.

The C-terminal and central-epitope antibodies of the present invention or a fragment of Aβ that induces such an antibody can also be used to treat patients having Alzheimer's disease and concurrent epilepsy. In animal models, compact plaques induce abnormal neuronal hyperactivity that can contribute to AD-specific epileptic seizures (Busche et al., Science 321:1686-1689 (2008)), axonal loss, dystrophic neurites, and neuronal injury and death. Shah et al., Am J Pathol 177:325-333 (2010); Sheng et al., J Neuropath Exp Neurol 57:714-717 (1998). Therefore, antibodies preferentially binding to compact plaques are particularly useful for Alzheimer's patients with concurrent epilepsy. The removal of compact plaques with central and C-terminal antibodies of the present invention can reduce symptoms of epileptic seizures in such patients as well as reducing or inhibiting further development of symptoms of Alzheimer's disease itself.

The C-terminal and central-epitope antibodies of the present invention can be used effectively in patients in any of the ApoE genotypes. In particular, the antibodies of the present invention can be used in patients with an E3/E3, E3/E4 or E4/E4 ApoE genotype.

### V. Treatment and Dosing

An “effective regime” is a dose, and frequency and route of administration that produces a desired result in a patient. In prophylactic applications, agents or pharmaceutical compositions or medicaments containing the same are administered to a patient susceptible to, or otherwise at risk of disease (e.g., Alzheimer's disease in a regime (dose, frequency and route of administration) effective to reduce the risk, lessen the severity, or delay the onset of at least one sign or symptom of the disease. In particular, the regime is preferably effective to reduce the amount of amyloid deposits (particularly compact plaques) or at least inhibit increase of the amount of amyloid deposits (particularly compact plaques) in the brain of the patient. Patients at risk of Alzheimer's disease include patients with above normal levels of amyloid deposits in the brain who have not been diagnosed with Alzheimer's disease and patients with mild cognitive impairment who have not been diagnosed with Alzheimer's disease. In therapeutic applications, agent compositions or medicaments are administered to a patient suspected of, or already suffering from Alzheimer's disease in a regime (dose, frequency and route of administration) effective to ameliorate or at least inhibit further deterioration of at least one sign or symptom of the disease. In particular, the regime is preferably effective to reduce or at least inhibit further increase of amyloid deposits (particularly compact plaques) in the patients.

Effective doses of the compositions of the present invention, for the treatment of the above described conditions vary depending upon many different factors, including means of administration, target site, physiological state of the patient, whether the patient is human or an animal, other medications administered, and whether treatment is prophylactic or therapeutic.

Optionally antibodies are administered to achieve a mean serum concentration of administered antibody of 0.1-60, 0.4-20, or 1-15 μg/ml in a patient. The serum concentration can be determined by actual measurement or predicted from standard pharmacokinetics (e.g., WinNonline Version 4.0.1 (Pharsight Corporation, Cary, USA)) based on the amount of antibody administered, frequency of administration, route of administration and antibody half-life.

The mean antibody concentration in the serum is optionally within a range of 1-10, 1-5 or 2-4 μg/ml. It is also optional to maintain a maximum serum concentration of the antibody in the patient less than about 28 μg antibody/ml serum for maximizing therapeutic benefit relative to the occurrence of possible side effects, particularly vascular edema. A preferred maximum serum concentration is within a range of about 4-28 μg antibody/ml serum. The combination of maximum serum less than about 28 μg antibody/ml serum and an mean serum concentration of the antibody in the patient is below about 7 μg antibody/ml serum is particularly beneficial. Optionally, the mean concentration is within a range of about 2-7 μg antibody/ml serum.

The concentration of Aβ in plasma following antibody administration changes roughly in parallel with changes of antibody serum concentration. In other words, plasma concentration of Aβ is highest after a dose of antibody and then declines as the concentration of antibody declines between doses. The dose and regime of antibody administration can be varied to obtain a desired level of Aβ in plasma. In such methods, the mean plasma concentration of antibody can be at least 450 pg/ml or for example, within a range of 600-3000 pg/ml or 700-2000 pg/ml or 800-1000 pg/ml.

Exemplary dosage ranges for antibodies are from about 0.01 to 10 mg/kg, 0.01 to 5 mg/kg, and in some cases from 0.1 to 3 mg/kg or 0.15-2 mg/kg or 0.15-1.5 mg/kg, of the host body weight. In some cases, the dose is 0.01, 0.05, 0.1, 0.25, 0.5, 0.75, 1, 1.5, 2, 2.5, 3, 3.5, 4, 4.5, 5, 6, 7, 8, 9, or 10 mg/kg. Subjects can be administered such doses daily, on alternative days, weekly, biweekly, monthly, quarterly, or according to any other schedule determined by empirical analysis. An exemplary treatment entails administration in multiple dosages over a prolonged period, for example, of at least three months, at least six months, at least nine months, or at least one year. Additional exemplary treatment regimes entail administration once per every two weeks or once a month or once every 3 to 6 months. The doses may be administered, for example, intravenously or subcutaneously.

For intravenous administration, doses of 0.1 mg/kg to 2 mg/kg, and preferably 0.5 mg/kg, 1.0 mg/kg or 1.5 mg/kg administered intravenously quarterly are suitable. Preferred doses of antibody for monthly intravenous administration occur in the range of 0.1-1.0 mg/kg antibody or preferably 0.5-1.0 mg/kg antibody.

For more frequent dosing, e.g., from weekly to monthly dosing, subcutaneous administration is preferred. Subcutaneous dosing is easier to administer and can reduce maximum serum concentrations relative to intravenous dosing. Exemplary doses for subcutaneous dosing are usually in the range of 0.01 to 0.6 mg/kg or 0.01-0.35 mg/kg, preferably, 0.05-0.25 mg/kg. For weekly or biweekly dosing, the dose is preferably in the range of 0.015-0.2 mg/kg, or 0.05-0.15 mg/kg. For weekly dosing, the dose is preferably 0.05 to 0.07 mg/kg, e.g., about 0.06 mg/kg. For biweekly dosing, the dose is preferably 0.1 to 0.15 mg/kg. For monthly dosing, the dose is preferably 0.1 to 0.3 mg/kg or about 0.2 mg/kg. Monthly dosing includes dosing by the calendar month or lunar month (i.e., every four weeks). Here as elsewhere in the application, dosages expressed in mg/kg can be converted to absolute mass dosages by multiplying by the mass of a typical patient (e.g., 70 or 75 kg) typically rounding to a whole number. Other regimes are described by e.g., PCT/US2007/009499. The dosage and frequency can be varied within these guidelines based on the ApoE status of the patient as discussed above.

The amount of an agent for active administration varies from 1-500 μg per patient and more usually from 5-100 μg per injection for human administration. Exemplary dosages per injection are 3, 10, 30, or 90 μg for each human injection. The mass of immunogen also depends on the mass ratio of immunogenic epitope within the immunogen to the mass of immunogen as a whole. Typically, 10⁻³ to 10⁻⁵ micromoles of immunogenic epitope are used for each immunization of immunogen. The timing of injections can vary significantly from once a day, to once a year, to once a decade. On any given day that a dosage of immunogen is given, the dosage is greater than 1 μg/patient and usually greater than 10 μg/patient if adjuvant is also administered, and greater than 10 μg/patient and usually greater than 100 μg/patient in the absence of adjuvant. A typical regimen consists of an immunization followed by booster injections at time intervals, such as 6 week intervals. Another regimen consists of an immunization followed by booster injections 1, 2 and 12 months later. Another regimen entails an injection every two months for life. Alternatively, booster injections can be on an irregular basis as indicated by monitoring of immune response. The dosage and frequency can be varied such that antibodies induced by an active agent have mean serum concentrations within a range of 0.1-60, 0.4-20, or 1-15 or 2-7 μg/ml as in passive administration.

### VI. Pharmaceutical Compositions

Agents (e.g., antibodies) of the invention are often administered as pharmaceutical compositions comprising an active therapeutic agent and a variety of other pharmaceutically acceptable components. See Remington's Pharmaceutical Science (15th ed., Mack Publishing Company, Easton, Pa. (1980)). The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

Pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes). Additionally, these carriers can function as immunostimulating agents (i.e., adjuvants).

Agents are typically administered parenterally. Antibodies are usually administered intravenously or subcutaneously. Agents for inducing an active immune response are usually administered subcutaneously or intramuscularly. For parenteral administration, agents of the invention can be administered as injectable dosages of a solution or suspension of the substance in a physiologically acceptable diluent with a pharmaceutical carrier that can be a sterile liquid such as water oils, saline, glycerol, or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin, for example, peanut oil, soybean oil, and mineral oil. In general, glycols such as propylene glycol or polyethylene glycol are preferred liquid carriers, particularly for injectable solutions. Antibodies can be administered in the form of a depot injection or implant preparation, which can be formulated in such a manner as to permit a sustained release of the active ingredient.

Typically, compositions are prepared as injectables, either as liquid solutions or suspensions in liquid vehicles prior to injection can also be prepared. The antibody can also be provided in lyophilized form for reconstitution before use. The antibody preparation also can be emulsified or encapsulated in liposomes or micro particles such as polylactide, polyglycolide, or copolymer for enhanced adjuvant effect, as discussed above (see Langer, Science 249: 1527 (1990) and Hanes, Advanced Drug Delivery Reviews 28:97 (1997)). The agents of this invention can be administered in the form of a depot injection or implant preparation, which can be formulated in such a manner as to permit a sustained or pulsatile release of the active ingredient.

Additional formulations suitable for other modes of administration include oral, intranasal, and pulmonary formulations, suppositories, and transdermal applications. For suppositories, binders and carriers include, for example, polyalkylene glycols or triglycerides; such suppositories can be formed from mixtures containing the active ingredient in the range of 0.5% to 10%, preferably 1%-2%. Oral formulations include excipients, such as pharmaceutical grades of mannitol, lactose, starch, magnesium stearate, sodium saccharine, cellulose, and magnesium carbonate. These compositions take the form of solutions, suspensions, tablets, pills, capsules, sustained release formulations or powders and contain 10%-95% of active ingredient, preferably 25%-70%.

### VII. Monitoring

Methods of in vivo imaging amyloid deposits in a patient can be used to diagnose or confirm a diagnosis or Alzheimer's disease, to identify the proportion of compact plaques or diffuse plaques relative to total plaques, or to monitor disease progression and/or response to treatment (e.g., clearance of compact plaques) in patients receiving an immunotherapy regime, as discussed herein). In some cases, monitoring includes evaluation of the size or relative proportion of compact plaques in the patent's brain relative to a prior scan of the same patient or relative to an historical control. A preferred imaging technique is PET scanning.

Recent advances in radioimaging have enabled the imaging of Aβ plaques in vivo in AD patients. Klunk and Mathis, Curr Opin Neurol 21:683-687 (2008). An exemplary compound for this approach is the positron emission tomography (PET) radiotracer “Pittsburgh Compound-B” (PiB), which binds with high affinity to aggregated Aβ (Id.), although other PET ligands are also available, as discussed in greater detail below. The in vivo retention of PiB in brains of people with AD shows a regional distribution that is very similar to the distribution of plaques observed post-mortem on histologically stained AD brain tissue. Ikonomovic et al., Brain 131:1630-1645 (2008). Using a fluorescent derivative of PiB, it has been shown that PiB binds preferentially to compact plaque on AD postmortem tissues, whereas diffuse plaques are less prominently labeled. Id. Thus, PET scanning can be used to identify AD patients with a high proportion of compact plaques relative to diffuse plaques.

A PET scan can be performed using, for example, a conventional PET imager and auxiliary equipment. The scan typically includes one or more regions of the brain known in general to be associated with deposits in Alzheimer's disease and one or more regions in which few if any deposits are generally present to serve as controls. Regions of the brain associated with presence of amyloid deposits in Alzheimer's disease include, for example, the anterior cingulated, posterior cingulated, frontal, temporal, parietal or occipital cortice of the brain. Regions of the brain associated with lack of deposits include, for example, subcortical white matter, pons, and the cerebellum.

Typically a baseline measurement is performed before commencing immunotherapy. One or more subsequent scans are then performed after commencing treatment. The first such scan after commencing treatment can be performed about 3-24 months after commencing treatment. Usually, such a scan is performed within 6-18 or 9-18 months of commencing treatments, such as for example, at about 6, 9, 12, 15 or 18 months. In some methods, a scan is performed 78 weeks after treatment. Any subsequent scans (i.e., 3^{rd} and subsequent scans) can be performed at intervals of, for example, quarterly, six-monthly, yearly or every two years.

After commencing immunotherapy, effects of immunotherapy on amyloid deposits can be first seen in the period of about 3-24 months, and more typically 6-18 months. The effect can be assessed as an overall decrease in amyloid deposits, a decrease in a type of amyloid deposits (e.g., compact plaques) or as a change in representation of compact plaques to diffuse plaques or ratios of compact or diffuse plaques to total plaques. Such changes are assessed relative to a baseline measurement before beginning immunotherapy. Such effects can be measured in one or more regions of the brain in which deposits are known to form or can be measured from an average of such regions. Decreases in overall amyloid, or compact plaques or ratio of compact plaques to diffuse plaques or proportion of compact plaques to total plaques can almost always be attributed as a treatment effect because amyloid deposits and the ratio of compact to diffuse plaques or proportion of compact plaques to total plaques do not usually decrease in the absence of treatment.

Maintenance of overall amyloid deposits or compact plaques or ratio of compact to diffuse plaques or compact plaques over total plaques at an approximately constant level or even a small increase in amyloid deposits can also be an indication of response to treatment albeit a suboptimal response. Such responses can be compared with a time course of levels of amyloid deposits in patients with Alzheimer's disease not receiving treatment to determine whether the immunotherapy is having an effect in inhibiting further increases of overall amyloid deposits, compact plaques or ratio of compact plaques to diffuse plaques.

The detected signal can be represented as a multidimensional image. The multidimensional image can be in two dimensions representing a cross-section through the brain, in three dimensions, representing the three dimensional brain or in four dimensions representing changes in the three dimensional brain over time. A color scale can be used with different colors indicating different amounts of label and inferentially amyloid deposit detected. The results of the scan can also be presented numerically with numbers relating to the amount of label detected and consequently amount of amyloid deposits. The label present in a region of the brain known to be associated with deposits in Alzheimer's disease can be compared with the label present in a region known not to be associated with deposits to provide a ratio indicative of the extent of deposits within the former region. For the same radiolabeled ligand, such ratios provide a comparable measure of amyloid deposits or compact plaques or ratio of compact to diffuse plaques and changes thereof between different patients

In some methods, a PET scan is performed concurrent with or in the same patient visit as an MRI or CAT scan. An MRI or CAT scan provides more anatomical detail of the brain than a PET scan. However, the image from a PET scan can be superimposed on an MRI or CAT scan image more precisely indicating the location of PET ligand and inferentially amyloid deposits relative to anatomical structures in the brain

PET ligands are usually administered to a patient to the systemic circulation by a peripheral route, with intravenous administration being preferred. PET ligands can thus be delivered by the systemic circulation across the blood brain barrier to come into contact with amyloid deposits. PET ligand binding to an amyloid deposit in the brain is immobilized and can be detected in a subsequent PET scan. Unbound PET ligand or PET ligand bound to soluble Aβ is cleared from the brain more rapidly than bound PET scan and is not detected or is detected to a lesser extent relative to the same amount of bound PET ligand.

Pittsburgh Compound-B ([¹¹C]PiB). (Klunk et al., Ann Neurol 55(3):306-319 (2004) Ikonomovic et al., Brain; 131:1630-1645 (2008)) is an exemplary PET ligand. PiB is thioflavin-analogue that binds to aggregated fibrillar deposits of the Aβ with low nanomolar affinity, enters the brain in amounts sufficient for imaging with PET, and clears rapidly from normal brain tissue. (Price et al., J. Cereb. Blood Flow Metab. 25:1528-1547 (2005)). At the low nanomolar concentrations typically used in PET studies, the binding of PiB to postmortem human brain has been shown to be selective for fibrillar Aβ deposits. (Ikonomovic et al., supra; Fodero-Tavoletti et al., J Neurosci; 27:10365-10371 (2007)). Compared with controls, AD patients show approximately two-fold retention of [¹¹C]PiB in areas of brain association cortex known pathologically to be targeted by Aβ deposits. [¹¹C]PiB retention is equivalent in AD patients and controls in areas known to be relatively unaffected by Aβ deposition (such as subcortical white matter, pons, and cerebellum). The dose of PET ligand administered can be measured by radioactivity. An exemplary dose, particularly for [¹¹C]PiB, is 12-18 μCi.

Other PET ligands that can be used include the Th-T PET ligand ¹⁸F-AH110690 (a 3′-fluoro analog of PIB from GE Healthcare, also known as flutemetamol); and two CR PET ligands: the stilbene derivative ¹⁸F-BAY94-9172 (Bayer Schering Pharma) (which performed comparably to ¹¹C-PIB in a preliminary study in AD and controls [Rowe, Lancet Neurol. 2008; 7(2):129-3535]), and (E)-4-(2-(6-(2-(2-(2-(2-18F-fluoroethoxy)ethoxy)ethoxy)pyridin-3-yl)vinyl)-N-methyl benzenamine (¹⁸F-AV-45 from Avid Radiopharmaeuticals) [Klunk, Curr Opin Neurol. 2008; 21(6):683-732, Rowe, supra, Nordberg, Neuropsychologia. 2008; 46(6):1636-41].

The interval between administering the PET ligand and performing the scan can depend on the PET ligand and particularly its rate of uptake and clearing into the brain, and the half-life of its radiolabel. The interval can be, for example, about 10-120 min or 30-90 min.

### VIII. Examples

### Examples 1-4

### Materials & Methods

Antibodies:

All antibodies were of the IgG1 isotype. Positive controls were 3D6 (Aβ₁₋₅) and 12A11 (Aβ₃₋₇). IgG1 was the negative control. C-terminal Aβ antibodies were 2G3 and 14C2 (for Aβ₄₀) and 21F12 (for Aβ₄₂).

Tissues:

PDAPP mice (Games et al., Nature 373:523-527 (1995) were obtained from an in-house colony. PSAPP mice (bigenic for doubly mutated hAPP and Presenilin 1, Holcomb et al., Nat Med 4:97-100 (1998); Gordon et al., Exp Neurol 173:183-195 (2002) were obtained from Dr. Steve Jacobson (Wyeth), and Line 41 mice (Swedish and London mutated hAPP with Thy1-promoter, (Rockenstein et al., J Neurosci Res 66:573-582 (2001)) were a generous gift from Dr. Eliezer Masliah (University of CA, San Diego). Mice were euthanized and brains were quickly removed and frozen on dry ice. Fresh-frozen tissues from AD patients were a generous gift from Dr. Elizabeth Head (Brain Bank at University of CA, Irvine).

Sectioning:

Ten micron cryo-cut sections were generated from fresh-frozen tissues using a Microm HM 550 cryo-cutter, and mounted onto Superfrost-Plus slides for immunostaining, or onto small, Vectabond-coated and autoclaved class coverslips for ex vivo assay. All sections were dried overnight at room temperature before any processing.

Sections for immunostaining were processed unfixed or fixed for 5 min in an acetone 75%/ethanol 25% mix, or in phosphate-buffered 4% paraformaldehyde.

Immunostaining:

For immunoperoxidase staining, primary antibodies were either unmodified or coupled to biotin, and used at a concentration of 2 and 3 μg/ml respectively. Secondary antibodies, when used, were from Vector, coupled to biotin, and used at 1:200 dilution. The ABC “Elite” peroxidase kit from Vector was used as a last step and antigen-bound antibodies were visualized using diaminobenzidine and H₂O₂ as peroxidase substrates. All sections were counterstained with Hematoxylin.

For immunofluorescent staining on sections mounted on Superfrost slides or on coverslips, primary antibodies were used at 1-2 μg/ml, and secondary antibodies at 1:200 dilution. Anti-Aβ antibodies were coupled to Alexa-594 (red fluorescence) using a kit from Molecular Probes. In addition to the above antibodies, the commercial antibody anti-CD11b (rat antibody, Serotec), was used for the detection of microglia after ex vivo assay, and was revealed by the use of an Alexa-488 (green fluorescence). Hoechst nuclear stain (blue fluorescence) was also used to reveal cell nuclei.

Ex Vivo Assay (Aβ Phagocytosis Assay):

This assay was performed as previously described in Bard et al., Nat Med 6:916-919 (2000). Briefly, fresh cryo-sections on coverslips were placed individually into wells of 24-well culture plates, and incubated with either a control antibody (3D6, IgG) or one C-terminal Aβ antibody (2G3, 14C2, or 21F12) at 3 μg/ml concentration. The antibodies were added to the sections 30 min prior to the addition of murine microglia, prepared a week before from mouse P1 pups, and added at a density of 5×10⁵ cells/well. After the addition of microglia, sections were incubated at 37° C., in a 5% CO₂ atmosphere, for 8 h-14 h. After that period, the antibody/microglia solution was aspirated from the sections, and the sections were fixed with phosphate-buffered 4%-paraformaledhyde before being used for immunostaining as described above.

### Example 1

### C-terminal Antibodies 2G3, 14C2, and 21F12 Recognize Plaques on Unfixed AD Brain Sections

The results of staining experiments on unfixed AD tissues (occipital cortex) is shown in FIG. 1 (panels A and B), and their semi-quantitative visual evaluation (done independently by 2 investigators; results averaged), in Table 2 (A and B), below. Unfixed sections were used to mimic exposure to Aβ antibodies in a patient undergoing therapy, i.e., the antibodies can interact with an antigen unmodified by fixation procedures that typically induce cross-linking (formalin or paraformaldehyde), or dehydration and precipitation (ethanol, acetone, methanol) of the proteins in the tissue section. 3D6 and 12A11 antibodies served as a positive control. No staining at all could be detected when using an irrelevant IgG (negative control) in the staining procedure. Each of the evaluated C-terminal antibodies bound to AD plaques, although not as strongly as 3D6 or 12A11. Binding of the C-terminal antibodies to AD plaques was observed in sections from AD patients of both apolipoprotein E3/E3 and apolipoprotein E3/E4 genotypes. These results show that C-terminal antibodies 2G3, 14C2, and 21F12 bind Aβ plaques.

TABLE 2
Semi-quantitative visual evaluation of AD tissue
staining with C-terminal epitope Aβ antibodies.

| | | | | | |
|---|---|---|---|---|---|
| Patient-# | | | | | |
| Braak Stage | 3D6 | 12A11 | 2G3 | 14C2 | 21F12 |

A (E3/E3 genotype)

| | | | | | |
|---|---|---|---|---|---|
| 05-02′ | ++++ | +++(+) | +++ | ++(+) | ++ |
| V | | | | | |
| 8-00′ | ++++ | ++++ | +++ | ++(+) | ++ |
| V | | | | | |
| 11-03 | ++++ | +++(+) | +++ | ++(+) | ++ |
| V | | | | | |
| 14-02 | ++++ | +++(+) | ++ | ++ | − |
| V | | | | | |
| 24-01 | ++++ | ++++ | +++(+) | +++(+) | +(+) |
| VI | | | | | |
| Median | ++++ | +++(+) | +++ | ++(+) | +(+) |

B (E3/E4 genotype)

| | | | | | |
|---|---|---|---|---|---|
| 05-02′ | ++++ | +++(+) | +++ | ++(+) | ++ |
| V | | | | | |
| 8-00′ | ++++ | ++++ | +++ | ++(+) | ++ |
| V | | | | | |
| 11-03 | ++++ | +++(+) | +++ | ++(+) | ++ |
| V | | | | | |
| 14-02 | ++++ | +++(+) | ++ | ++ | − |
| V | | | | | |
| 24-01 | ++++ | ++++ | +++(+) | +++(+) | +(+) |
| VI | | | | | |
| Median | ++++ | +++(+) | +++ | ++(+) | +(+) |

### Example 2

### C-terminal Antibodies 2G3, 14C2, and 21F12 Recognize Plaques on Unfixed and Fixed Sections of PSAPP and Line 41, but Not PDAPP Mice

To investigate the binding of C-terminal Aβ antibodies 2G3, 14C2, and 21F12 on sections of hAPP transgenic mice, cryo-sections of the following models were generated (3 mice/genotype, 1-2 section/mouse for each antibody and staining condition): PDAPP (homozygous mice, 24-month-old); PSAPP (heterozygous, 13-month-old); and Line 41 (heterozygous, 18-month-old).

Two different fixation conditions (acetone 75%/ethanol 25% mix, 4% phosphate-buffered paraformaldehyde) we compared to unfixed sections. All Aβ antibodies were biotinylated. Antibody 3D6 served as a control, and IgG as a negative control. No signal was seen with IgG (not shown).

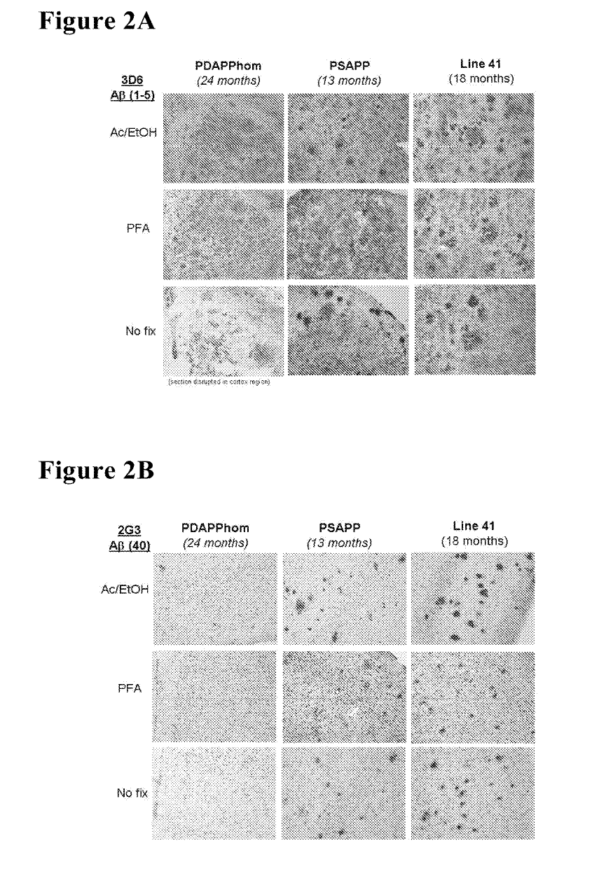

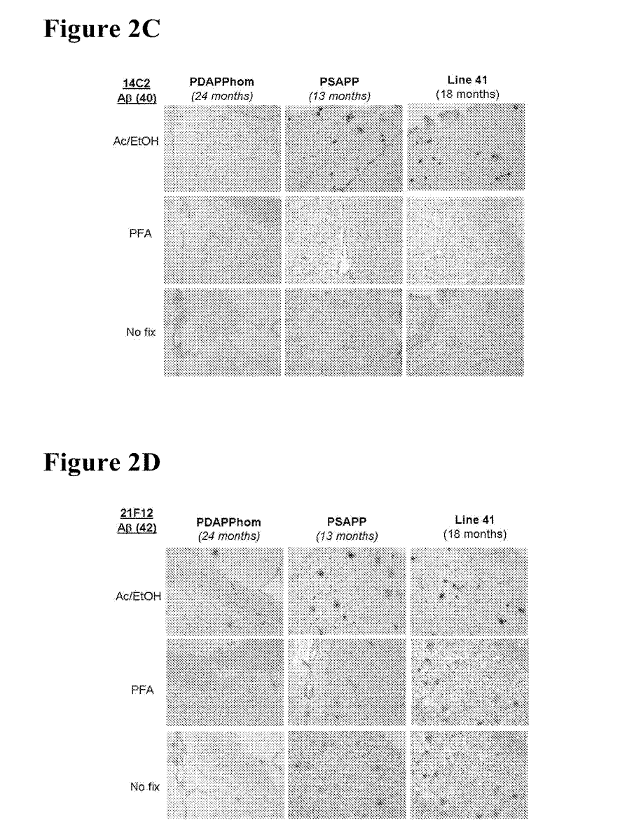

Results are shown in FIG. 2 for the frontal cortex of the 3 mouse models (A: 3DF6, B: 2G3, C: 14C2, D: 21F12). 3D6 labeled plaques heavily in all models and under all conditions. Compared to 3D6, C-terminal antibodies only labeled a small subset of plaques. The Aβ(40) antibodies 2G3 and 14C2 labeled plaques in PSAPP and Line 41 only, with 2G3 labeling plaques to a similar extent in all three conditions, whereas 14C2 labeled only after acetone/ethanol fixation. The Aβ(42) antibody 21F12 labeled a small number of plaques in PDAPP mice, and a larger number in PSAPP and Line 41 mice. Staining with this antibody was somewhat stronger after acetone/ethanol fixation.

These results indicate that Aβ(40) and Aβ(42) antibodies recognize plaques in some mouse models such as PSAPP and Line 41 mice.

### Example 3

### C-terminal Antibodies Bind Primarily to Plaques With Compact Appearance in AD and PSAPP Mice

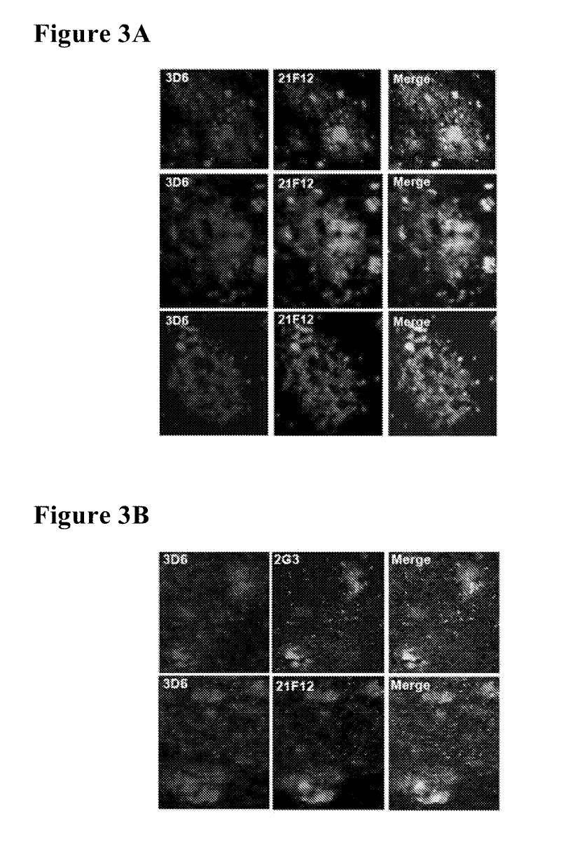

To determine the overlap between plaque binding of 3D6 and that of C-terminal Aβ antibodies, fluorescent double-labeling stainings were performed on unfixed AD and PSAPP sections. Results are shown in FIG. 3. Double staining gave interpretable results only for double labeling of 3D6+21F12 in AD, and for 3D6 and 2G3 or 3D6 and 21F12 in PSAPP sections.

The results show that 21F12 binds strongly to the dense core of plaques in AD sections (FIG. 3A, upper and middle rows), and to a lesser extent to plaques with a more diffuse appearance (FIG. 3A, lower row). In PSAPP mouse sections, 2G3 and 21F12 bind primarily to the dense core of plaques (FIG. 3B).

### Example 4

### C-terminal Antibodies Promote Microglial Phagocytosis of Aβ Plaques from PSAPP and Line 41 Mouse Sections in ex vivo Assay

To determine whether C-terminal Aβ antibodies are able to clear plaques, we used them in the ex vivo assay on cryo-sections from PSAPP and Line 41 mice.

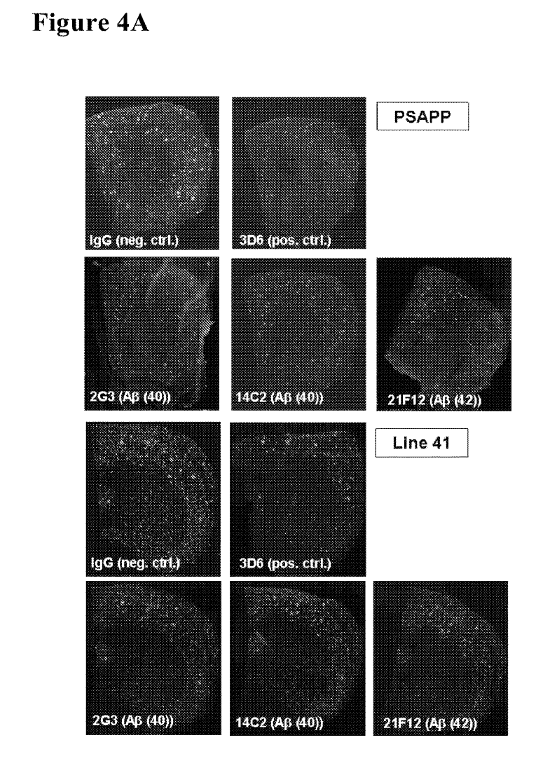

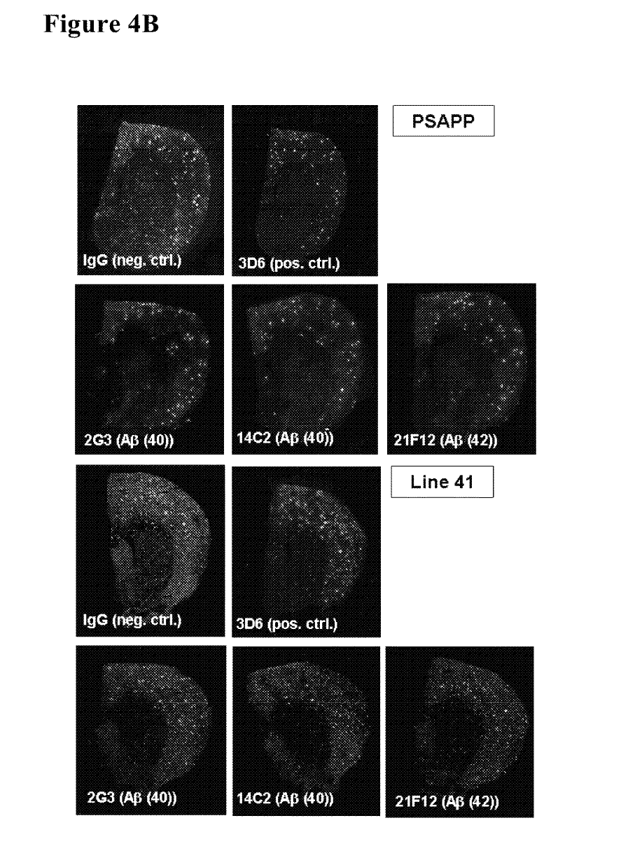

Sections from hemibrains were incubated with one Aβ antibody (3D6, 2G3, 14C2, 21F12, all IgG1 isotype) or with isotype-matched IgG negative control, and primary murine microglia as described above in the Materials and Methods. The sections were then triple-stained with 3D6 (red channel), the microglial marker CD11b (green channel), and the nuclear stain Hoechst (blue channel). Subsequently, to determine if the Aβ antibodies elicited plaque clearance, the red channel signals (3D6, for plaques) from whole sections were digitally scanned with a Retiga camera (QImaging) coupled to an Olympus BX61 microscope using a 10× objective. Images of whole sections were digitally reconstituted in black & white using the Metamorph® software, to show the plaque signals revealed by 3D6 staining. Results of two such experiments are shown in FIGS. 4A and 4B. The results show that all Aβ antibodies lead to a decrease in plaque signal, indicating that they all can clear plaques in the ex vivo assay.

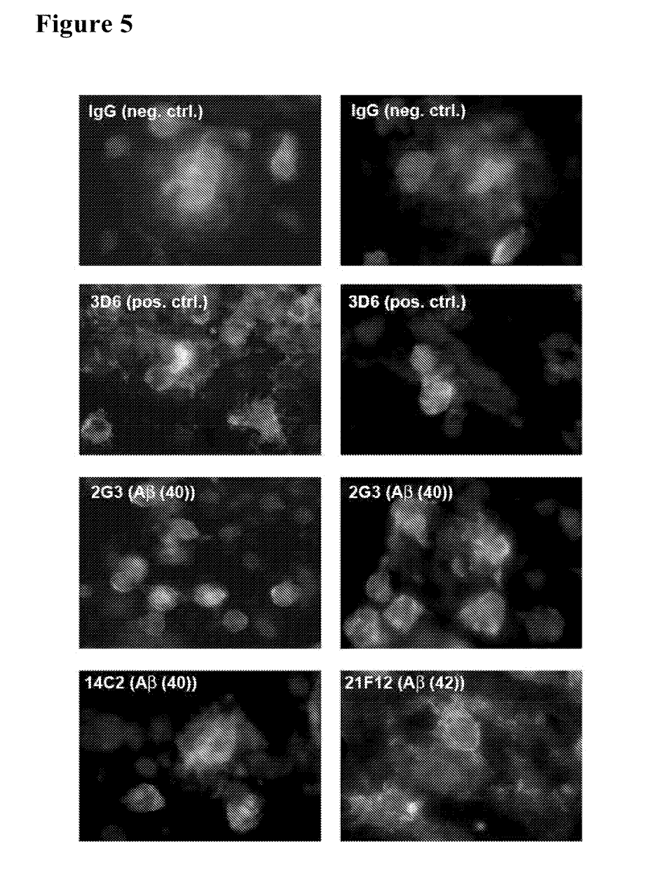

To demonstrate that microglial phagocytosis is involved in these ex vivo experiments with C-terminal Aβ antibodies, sections were then viewed at high power (40× objective), and all three channels (red, green, and blue) were imaged simultaneously. Results, showing the presence of Aβ inside microglia for the 3D6, 2G3, 14C2 and 21F12 antibodies, are shown in FIG. 5.

### Examples 5-8

### Materials & Methods

Antibodies:

All antibodies were of the IgG1 isotype. 3D6 (Aβ₁₋₅) was the positive control. IgG1 was the negative control. Central-epitope Aβ antibodies were 266 (Aβ₁₆₋₂₃). 15C11 (Aβ₁₈₋₂₂), and 22D12 (Aβ₁₈₋₂₂).

Tissues:

PDAPP mice (Games et al. (1995), supra) were obtained from an in-house colony. PSAPP mice (bigenic for doubly mutated hAPP and Presenilin 1, (Holcomb et al. (1998), supra; Gordon et al. (2002), supra) were obtained from Dr. Steve Jacobson (Wyeth). Mice were euthanized by CO₂ exposure, brains were quickly extracted and frozen on dry ice. Fresh-frozen tissues from AD patients were a generous gift from Dr. Elizabeth Head (Brain Bank at University of CA, Irvine).

Sectioning:

Ten micron cryo-cut sections were generated from fresh-frozen tissues using a Microm HM 550 cryo-cutter, and mounted onto Superfrost-Plus® slides for immunostaining, or onto small, Vectabone-coated and autoclaved class coverslips for ex vivo assay. All sections were dried overnight at room temperature before any processing. Sections for immunostaining were processed unfixed.

Immunostaining:

For immunoperoxidase staining, primary antibodies were used at a concentration of 3 μg/ml. Secondary antibodies were from Vector, coupled to biotin, and used at 1:200 dilution. The ABC “Elite” peroxidase kit from Vector was used as a last step and antigen-bound antibodies were visualized using diaminobenzidine and H₂O₂ as peroxidase substrates. AD sections were counterstained with Hematoxylin.

For immunofluorescent staining on sections mounted on Superfrost slides or on coverslips, primary antibodies were used at 1-2 μg/ml, and secondary antibodies at 1:200 dilution. Anti-Aβ antibodies were coupled to Alexa-594 (red fluorescence) using a kit from Molecular Probes. In addition to the above antibodies, the commercial antibody anti-CD11b (rat antibody, Serotec) was used for the detection of microglia after ex vivo assay, and was revealed by the use of an Alexa-488 (green fluorescence). Hoechst nuclear stain (blue fluorescence) was also used to reveal cell nuclei.

Ex Vivo Assay (Aβ Phagocytosis Assay):

This assay was performed as previously described in Bard et al. (2000), supra. Briefly, fresh cryo-sections on coverslips were placed individually into wells of 24-well culture plates, and incubated with either a control antibodies (3D6, IgG) or one central Aβ antibody (266, 15C11, or 22D12) at 3 μg/ml concentration. The antibodies were added to the sections 30 min prior to the addition of murine microglia, prepared a week before from mouse P1 pups, and added at a density of 5×10⁵ cells/well. After the addition of microglia, sections were incubated at 37° C., in a 5% CO₂ atmosphere, for 8 h-14 h. After that period, the antibody/microglia solution was aspirated from the sections, and the sections were fixed with phosphate-buffered 4%-paraformaledhyde before being used for immunostaining as described above.

### Example 5

### Central-epitope Antibodies 266, 15C11, and 22D12 Recognize Plaques on Unfixed AD Sections

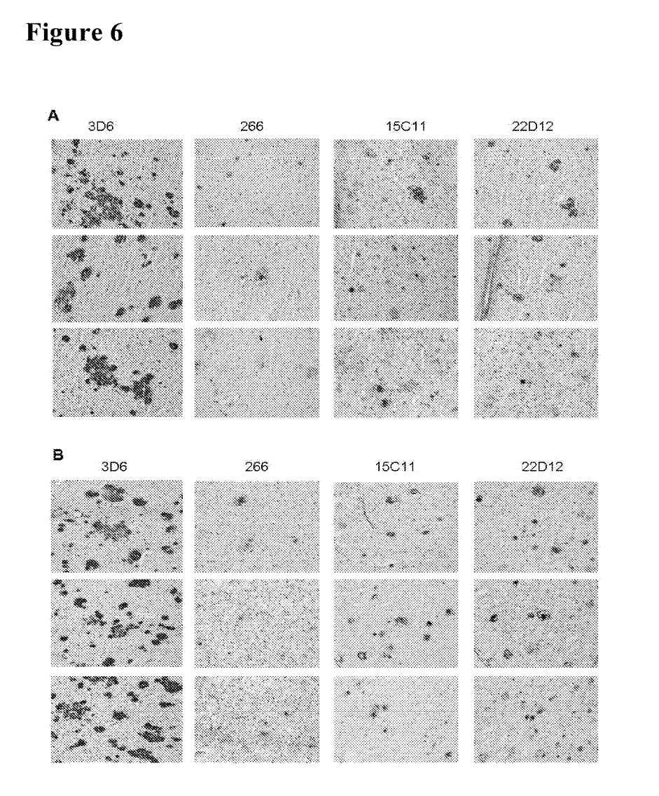

The results of staining experiments on unfixed AD tissues (occipital cortex) is shown in FIGS. 6 A, B, and their semi-quantitative visual evaluation (done independently by 2 investigators; results averages), in Table 3 (A and B), below. Unfixed sections were used to mimic exposure to Aβ antibodies in a patient undergoing therapy, i.e., the antibodies can interact with an antigen unmodified by fixation procedures that typically induce cross-linking (formalin or paraformaldehyde), or dehydration and precipitation (ethanol, acetone, methanol) of the proteins in the tissue section. 3D6 antibody served as a positive control. No staining at all could be detected when using an irrelevant IgG (negative control) in the staining procedure. Each of the evaluated central-epitope Aβ antibodies bound to AD plaques, although not as strongly as 3D6. Binding of the central-epitope antibodies to AD plaques was observed in sections from AD patients of both apolipoprotein E3/E3 and apolipoprotein E3/E4 genotypes. These results show that central-epitope Aβ antibodies 266, 15C11, and 22D12 bind Aβ plaques.

TABLE 3
Semi-quantitative visual evaluation of AD tissue
staining with central-epitope Aβ antibodies.

| Patient-# | | | | |
|---|---|---|---|---|
| Braak Stage | 3D6 | 266 | 15C11 | 22D12 |

A (E3/E3 genotype)

| | | | | |
|---|---|---|---|---|
| 01-02 | ++++ | + | + | ++ |
| VI | | | | |
| 04-02 | ++++ | +/− | + | +(+) |
| VI | | | | |
| 10-02 | ++++ | + | ++ | ++ |
| VI | | | | |
| 16-02 | ++++ | + | ++ | ++ |
| V | | | | |
| 26-00 | ++++ | +/− | +/− | +/− |
| VI | | | | |
| Median | ++++ | + | ++ | ++ |

B (E3/E4 genotype)

| | | | | |
|---|---|---|---|---|
| 05-02′ | ++++ | + | ++ | +(+) |
| V | | | | |
| 8-00′ | ++++ | ++ | ++(+) | ++(+) |
| V | | | | |
| 11-03 | ++++ | + | ++ | ++ |
| V | | | | |
| 14-02 | ++++ | + | ++ | ++ |
| V | | | | |
| 24-01 | ++++ | + | ++(+) | +++ |
| VI | | | | |
| Median | ++++ | + | ++ | ++ |

### Example 6

### Central-epitope Aβ Antibodies 266, 15C11, and 22D12 Recognize Plaques on Unfixed Sections of PSAPP, but Not PDAPP Mice

To investigate the binding of central-epitope Aβ antibodies 266, 15C11, and 22D12 on sections of hAPP transgenic mice, cryo-sections of the following two models were generated (3 mice/genotype, 1-2 section/mouse for each antibody and staining condition): PDAPP (hemizygote mice, 20-month-old); and PSAPP (heterozygous, 13-month-old).

Sections were processed unfixed as described above in the Materials and Methods. No signal was seen with IgG (not shown). Non-specific background was observed in sections of non-transgenic control mice.

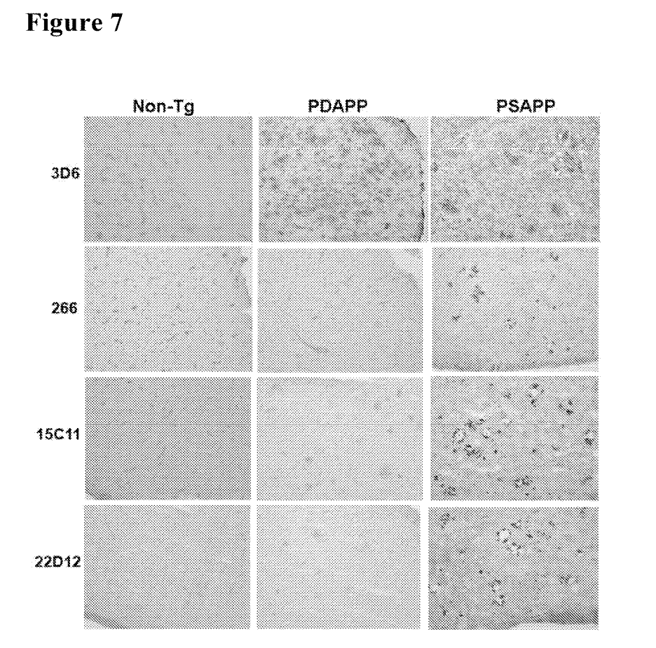

Results are shown in FIG. 7 for the frontal cortex of the 2 mouse models. 3D6 labeled plaques heavily in all models. Central-epitope Aβ antibodies labeled a subset of the plaques labeled by 3D6. These results indicate that central-epitope Aβ antibodies recognize plaques in some mouse models such as PSAPP.

### Example 7

### Central-epitope Antibodies Bind Primarily to Compact Plaques in AD and PSAPP Mice

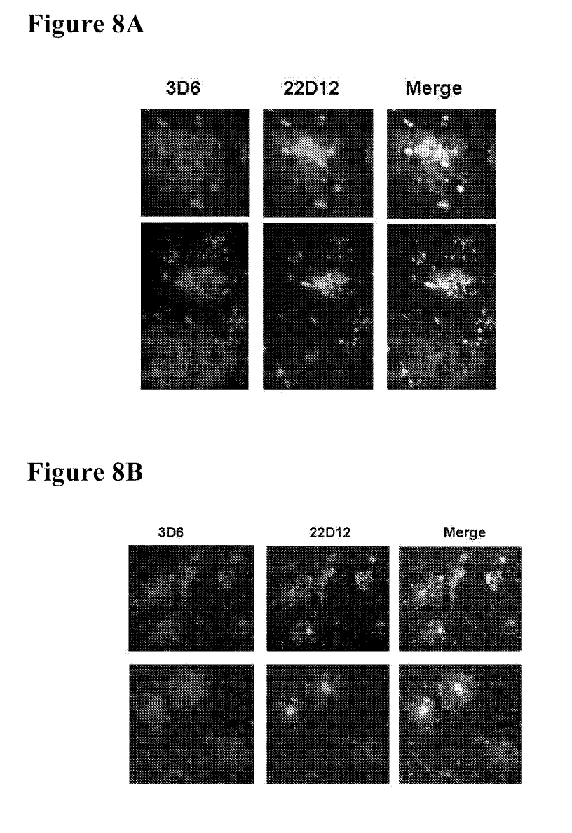

To determine the overlap between plaque binding of 3D6 and that of central-epitope Aβ antibodies, fluorescent double-labeling stainings were performed on unfixed AD and PSAPP sections using 3D6 in combination with 22D12. Results are shown in FIGS. 8A and 8B.

The results show that 22D12 binds strongly to the dense core of plaques in AD and PSAPP sections.

### Example 8

### Central-epitope Aβ Antibodies Promote Microglial Phagocytosis of Aβ Plaques from PSAPP Mouse, but Not PDAPP, Sections in Ex Vivo Assay

To determine whether central-epitope Aβ antibodies are able to clear plaques, we used the 266, 15C11, and 22D12 antibodies in the ex vivo assay on cryo-sections from PDAPP and PSAPP mice.

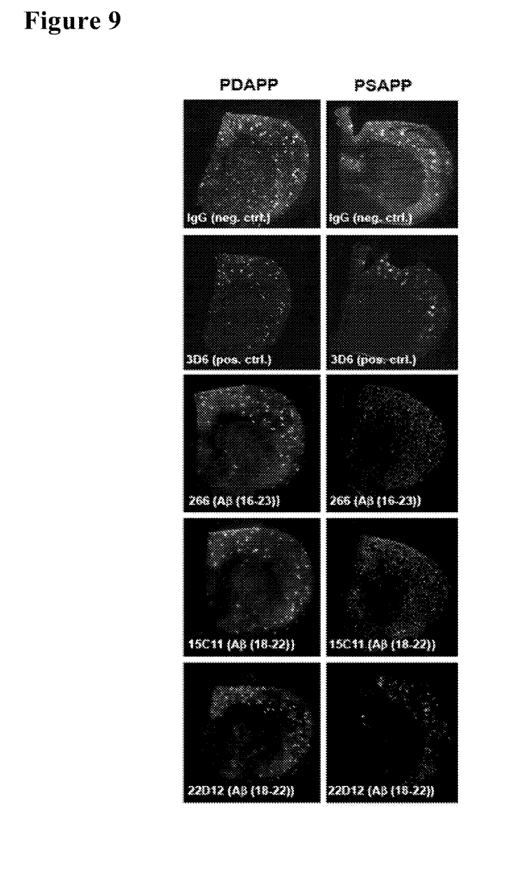

Sections from hemibrains were incubated with an Aβ antibody (3D6 (positive control) or 266 (Aβ₁₆₋₂₃), 15C11 (Aβ₁₈₋₂₂), 22D12 (Aβ₁₈₋₂₂), all IgG1 isotype) antibodies, or with isotype-matched IgG negative control, and primary murine microglia as described above in the Materials and Methods. The sections were then triple-stained with 3D6 (red channel), the microglial marker CD11b (green channel), and the nuclear stain Hoechst (blue channel). Subsequently, to determine if the Aβ antibodies elicited plaque clearance, the red channel signals (3D6, for plaques) from whole sections were digitally scanned with a Retiga camera (QImaging) coupled to an Olympus BX61 microscope using a 10× objective. Images of whole sections were digitally reconstituted in black & white using the Metamorph® software, to show the plaque signals revealed by 3D6 staining. Results of such an experiment are shown in FIG. 9. The results show that only the positive control, 3D6, decreases the plaque signal in PDAPP mouse sections, but all Aβ antibodies lead to a decrease in plaque signal in PSAPP sections, indicating that central-epitope Aβ antibodies can clear plaques in PSAPP, but not PDAPP mice.

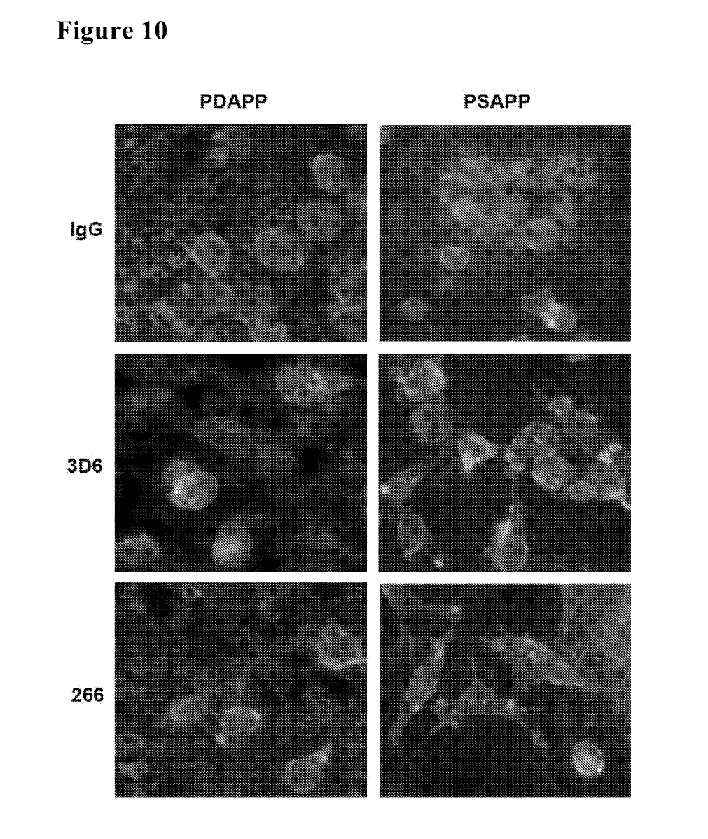

To demonstrate that microglial phagocytosis is implicated in clearing plaques in these ex vivo experiments with central-epitope Aβ antibodies, sections (with negative control IgG, positive control 3D6, or 266 antibodies), were then viewed at high power (40× objective), and all three channels (red, green, and blue) were imaged simultaneously. Results are shown in FIG. 10. Microglial phagocytosis of Aβ was observed after ex vivo with 3D6 in sections from both mouse models (PDAPP and PSAPP), but only in sections of PSAPP mouse after ex vivo with the central-epitope antibody 266.

All references cited herein, including patents, patent applications, journal articles, websites, accession numbers and the like are incorporated herein by reference in their entireties for all purposes to the same extent as if so individually denoted. Unless otherwise apparent from the context any step, element, feature, embodiment or aspect of the invention can be used in combination with any other. Although this invention has been described in connection with specific embodiments thereof, it is capable of further modifications. This application is intended to cover any variations, uses, or adaptations of the invention following, in general, the principles of the invention and including such departures from the present disclosure as come within known or customary practice within the art to which the invention pertains and as may be applied to the essential features hereinbefore set forth.

What is claimed is:

1. A method of treating a patient diagnosed with mid- or late-stage Alzheimer's disease, comprising administering to the patient an effective regime of an antibody that binds to an epitope within residues 12-43 of Aβ and preferentially binds compact plaques relative to diffuse plaques.

2. The method of claim 1, wherein the patient has been diagnosed with mid-stage Alzheimer's disease.

3. The method of claim 1, wherein the patient has been diagnosed with late-stage Alzheimer's disease.

4. The method of claim 1, wherein the antibody has specificity for a central epitope of Aβ.

5. The method of claim 4, wherein the antibody is a 266 antibody or a chimeric, humanized or veneered form thereof, a 15C11 antibody or a chimeric, humanized or veneered form thereof, or a 22D12 antibody or a chimeric, humanized or veneered form thereof.

6. The method of claim 5, wherein the antibody comprises: three light chain variable region complementarity determining regions (CDRs), wherein CDR L1 comprises the amino acid sequence of SEQ ID NO:4, CDR L2 comprises the amino acid sequence of SEQ ID NO:5, and CDR L3 comprises the amino acid sequence of SEQ ID NO:6, and three heavy chain variable region CDRs, wherein CDR H1 comprises the amino acid sequence of SEQ ID NO:7, CDR H2 comprises the amino acid sequence of SEQ ID NO:8, and CDR H3 comprises the amino acid sequence of SEQ ID NO:9.

7. The method of claim 5, wherein the antibody comprises: three light chain variable region CDRs, wherein CDR L1 comprises the amino acid sequence of residues 24 to 39 of SEQ ID NO:14, CDR L2 comprises the amino acid sequence of residues 55 to 61 of SEQ ID NO:14, and CDR L3 comprises the amino acid sequence of residues 94 to 101 of SEQ ID NO:14, and three heavy chain variable region CDRs, wherein CDR H1 comprises the amino acid sequence of residues 26 to 35 of SEQ ID NO:15, CDR H2 comprises the amino acid sequence of residues 50 to 66 SEQ ID NO:15, and CDR H3 comprises the amino acid sequence of residues 99 to 101 of SEQ ID NO:15.

8. The method of claim 5, wherein the antibody comprises: three light chain variable region CDRs of 22D12, and three heavy chain variable region CDRs of 22D12.

9. The method of claim 1, wherein the antibody has specificity for a C-terminal epitope of Aβ.

10. The method of claim 9, wherein the antibody is a 2G3 antibody or a chimeric, humanized or veneered form thereof, a 14C2 antibody or a chimeric, humanized or veneered form thereof, or a 21F12 antibody or a chimeric, humanized or veneered form thereof.

11. The method of claim 10, wherein the antibody comprises: three light chain variable region complementarity determining regions (CDRs) of 2G3, and three heavy chain variable region CDRs of 2G3.

12. The method of claim 10, wherein the antibody comprises: three light chain variable region CDRs of 14C2, and three heavy chain variable region CDRs of 14C2.

13. The method of claim 10, wherein the antibody comprises: three light chain variable region CDRs, wherein CDR L1 comprises the amino acid sequence of residues 24 to 39 of SEQ ID NO:3, CDR L2 comprises the amino acid sequence of residues 55 to 61 of SEQ ID NO:3, and CDR L3 comprises the amino acid sequence of residues 94 to 102 of SEQ ID NO:3, and three heavy chain variable region CDRs, wherein CDR H1 comprises the amino acid sequence of residues 26 to 35 of SEQ ID NO:2, CDR H2 comprises the amino acid sequence of residues 50 to 66 of SEQ ID NO:2, and CDR H3 comprises the amino acid sequence of residues 99 to 106 of SEQ ID NO:2.

14. The method of any one of claims 1 to 13, wherein the antibody is a chimeric antibody or a humanized antibody.

15. The method of claim 14, wherein the antibody is a humanized antibody.

16. The method of claim 14, wherein the antibody is of the IgG1 subtype.

17. A method of treating a patient diagnosed with Alzheimer's disease and having a greater proportion of compact plaques than diffuse plaques relative to total plaques, comprising administering to the patient an effective regime of an antibody that binds to an epitope within residues 12-43 of Aβ and preferentially binds compact plaques relative to diffuse plaques.

18. The method of claim 17, wherein the proportion of compact plaques is at least 40% of total plaques.

19. The method of claim 17, wherein the proportions of compact and diffuse plaques relative to total plaques are determined by positron emission tomography (PET) scanning.

20. The method of claim 19, wherein the PET scanning comprises detecting a PET ligand selected from the group consisting of [¹⁸F]AV-14, [¹⁸F]AV-144, [¹¹C]AZD2995, [¹⁸F]-AZD4694 and [¹⁸F]SMIBR-W372.

21. The method of claim 17, wherein the antibody has specificity for a central epitope of Aβ.

22. The method of claim 21, wherein the antibody is a 266 antibody or a chimeric, humanized or veneered form thereof, a 15C11 antibody or a chimeric, humanized or veneered form thereof, or a 22D12 antibody or a chimeric, humanized or veneered form thereof.

23. The method of claim 22, wherein the antibody comprises: three light chain variable region complementarity determining regions (CDRs), wherein CDR L1 comprises the amino acid sequence of SEQ ID NO:4, CDR L2 comprises the amino acid sequence of SEQ ID NO:5, and CDR L3 comprises the amino acid sequence of SEQ ID NO:6, and three heavy chain variable region CDRs, wherein CDR H1 comprises the amino acid sequence of SEQ ID NO:7, CDR H2 comprises the amino acid sequence of SEQ ID NO:8, and CDR H3 comprises the amino acid sequence of SEQ ID NO:9.

24. The method of claim 22, wherein the antibody comprises: three light chain variable region CDRs, wherein CDR L1 comprises the amino acid sequence of residues 24 to 39 of SEQ ID NO:14, CDR L2 comprises the amino acid sequence of residues 55 to 61 of SEQ ID NO:14, and CDR L3 comprises the amino acid sequence of residues 94 to 101 of SEQ ID NO:14, and three heavy chain variable region CDRs, wherein CDR H1 comprises the amino acid sequence of residues 26 to 35 of SEQ ID NO:15, CDR H2 comprises the amino acid sequence of residues 50 to 66 SEQ ID NO:15, and CDR H3 comprises the amino acid sequence of residues 99 to 101 of SEQ ID NO:15.

25. The method of claim 22, wherein the antibody comprises: three light chain variable region CDRs of 22D12, and three heavy chain variable region CDRs of 22D12.

26. The method of claim 17, wherein the antibody has specificity for a C-terminal epitope of Aβ.

27. The method of claim 26, wherein the antibody is a 2G3 antibody or a chimeric, humanized or veneered form thereof, a 14C2 antibody or a chimeric, humanized or veneered form thereof, or a 21F12 antibody or a chimeric, humanized or veneered form thereof.

28. The method of claim 27, wherein the antibody comprises: three light chain variable region complementarity determining regions (CDRs) of 2G3, and three heavy chain variable region CDRs of 2G3.

29. The method of claim 27, wherein the antibody comprises: three light chain variable region CDRs of 14C2, and three heavy chain variable region CDRs of 14C2.

30. The method of claim 27, wherein the antibody comprises: three light chain variable region CDRs, wherein CDR L1 comprises the amino acid sequence of residues 24 to 39 of SEQ ID NO:3, CDR L2 comprises the amino acid sequence of residues 55 to 61 of SEQ ID NO:3, and CDR L3 comprises the amino acid sequence of residues 94 to 102 of SEQ ID NO:3, and three heavy chain variable region CDRs, wherein CDR H1 comprises the amino acid sequence of residues 26 to 35 of SEQ ID NO:2, CDR H2 comprises the amino acid sequence of residues 50 to 66 of SEQ ID NO:2, and CDR H3 comprises the amino acid sequence of residues 99 to 106 of SEQ ID NO:2.

31. The method of any one of claims 17 to 30, wherein the antibody is a chimeric antibody or a humanized antibody.

32. The method of claim 31, wherein the antibody is a humanized antibody.

33. The method of claim 31, wherein the antibody is of the IgG1 subtype.

34. A method of treating a patient diagnosed with Alzheimer's disease and having symptoms of epileptic seizures, comprising administering to the patient an effective regime of an antibody that binds to an epitope within residues 12-43 of Aβ and preferentially binds compact plaques relative to diffuse plaques.

35. The method of claim 34, wherein total amyloid plaque burden and the symptoms of epileptic seizures are reduced.

36. The method of claim 34, wherein the antibody has specificity for a central epitope of β-amyloid.

37. The method of claim 36, wherein the antibody is a 266 antibody or a chimeric, humanized or veneered form thereof, a 15C11 antibody or a chimeric, humanized or veneered form thereof, or a 22D12 antibody or a chimeric, humanized or veneered form thereof.

38. The method of claim 37, wherein the antibody comprises: three light chain variable region complementarity determining regions (CDRs), wherein CDR L1 comprises the amino acid sequence of SEQ ID NO:4, CDR L2 comprises the amino acid sequence of SEQ ID NO:5, and CDR L3 comprises the amino acid sequence of SEQ ID NO:6, and three heavy chain variable region CDRs, wherein CDR H1 comprises the amino acid sequence of SEQ ID NO:7, CDR H2 comprises the amino acid sequence of SEQ ID NO:8, and CDR H3 comprises the amino acid sequence of SEQ ID NO:9.

39. The method of claim 37, wherein the antibody comprises: three light chain variable region CDRs, wherein CDR L1 comprises the amino acid sequence of residues 24 to 39 of SEQ ID NO:14, CDR L2 comprises the amino acid sequence of residues 55 to 61 of SEQ ID NO:14, and CDR L3 comprises the amino acid sequence of residues 94 to 101 of SEQ ID NO:14, and three heavy chain variable region CDRs, wherein CDR H1 comprises the amino acid sequence of residues 26 to 35 of SEQ ID NO:15, CDR H2 comprises the amino acid sequence of residues 50 to 66 SEQ ID NO:15, and CDR H3 comprises the amino acid sequence of residues 99 to 101 of SEQ ID NO:15.

40. The method of claim 37, wherein the antibody comprises: three light chain variable region CDRs of 22D12, and three heavy chain variable region CDRs of 22D12.

41. The method of claim 34, wherein the antibody has specificity for a C-terminal epitope of Aβ.

42. The method of claim 41, wherein the antibody is a 2G3 antibody or a chimeric, humanized or veneered form thereof, a 14C2 antibody or a chimeric, humanized or veneered form thereof, or a 21F12 antibody or a chimeric, humanized or veneered form thereof.

43. The method of claim 42, wherein the antibody comprises: three light chain variable region complementarity determining regions (CDRs) of 2G3, and three heavy chain variable region CDRs of 2G3.

44. The method of claim 42, wherein the antibody comprises: three light chain variable region CDRs of 14C2, and three heavy chain variable region CDRs of 14C2.

45. The method of claim 42, wherein the antibody comprises: three light chain variable region CDRs, wherein CDR L1 comprises the amino acid sequence of residues 24 to 39 of SEQ ID NO:3, CDR L2 comprises the amino acid sequence of residues 55 to 61 of SEQ ID NO:3, and CDR L3 comprises the amino acid sequence of residues 94 to 102 of SEQ ID NO:3, and three heavy chain variable region CDRs, wherein CDR H1 comprises the amino acid sequence of residues 26 to 35 of SEQ ID NO:2, CDR H2 comprises the amino acid sequence of residues 50 to 66 of SEQ ID NO:2, and CDR H3 comprises the amino acid sequence of residues 99 to 106 of SEQ ID NO:2.

46. The method of any one of claims 34 to 45, wherein the antibody is a chimeric antibody or a humanized antibody.

47. The method of claim 46, wherein the antibody is a humanized antibody.

48. The method of claim 46, wherein the antibody is of the IgG1 subtype.

49. A method of treating a patient diagnosed with Alzheimer's disease, comprising:
(a) administering to the patient an effective regime of an antibody that preferentially binds compact plaques relative to diffuse plaques, wherein the antibody has specificity for a central or C-terminal epitope of Aβ; and
(b) monitoring one or more attributes of compact plaques in the patient's brain using PET scanning.

50. The method of claim 49, wherein the one or more attributes of the compact plaques is identified using radiotracer PiB.

51. The method of claim 49, wherein the one or more attributes comprise a reduction in size of one or more compact plaques relative to a prior PET scan.

52. The method of claim 49, wherein the antibody has specificity for a central epitope of Aβ.

53. The method of claim 52, wherein the antibody is a 266 antibody or a chimeric, humanized or veneered form thereof, a 15C11 antibody or a chimeric, humanized or veneered form thereof, or a 22D12 antibody or a chimeric, humanized or veneered form thereof.

54. The method of claim 53, wherein the antibody comprises: three light chain variable region complementarity determining regions (CDRs), wherein CDR L1 comprises the amino acid sequence of SEQ ID NO:4, CDR L2 comprises the amino acid sequence of SEQ ID NO:5, and CDR L3 comprises the amino acid sequence of SEQ ID NO:6, and three heavy chain variable region CDRs, wherein CDR H1 comprises the amino acid sequence of SEQ ID NO:7, CDR H2 comprises the amino acid sequence of SEQ ID NO:8, and CDR H3 comprises the amino acid sequence of SEQ ID NO:9.

55. The method of claim 53, wherein the antibody comprises: three light chain variable region CDRs, wherein CDR L1 comprises the amino acid sequence of residues 24 to 39 of SEQ ID NO:14, CDR L2 comprises the amino acid sequence of residues 55 to 61 of SEQ ID NO:14, and CDR L3 comprises the amino acid sequence of residues 94 to 101 of SEQ ID NO:14, and three heavy chain variable region CDRs, wherein CDR H1 comprises the amino acid sequence of residues 26 to 35 of SEQ ID NO:15, CDR H2 comprises the amino acid sequence of residues 50 to 66 SEQ ID NO:15, and CDR H3 comprises the amino acid sequence of residues 99 to 101 of SEQ ID NO:15.

56. The method of claim 53, wherein the antibody comprises: three light chain variable region CDRs of 22D12, and three heavy chain variable region CDRs of 22D12.

57. The method of claim 49, wherein the antibody has specificity for a C-terminal epitope of Aβ.

58. The method of claim 57, wherein the antibody is a 2G3 antibody or a chimeric, humanized or veneered form thereof, a 14C2 antibody or a chimeric, humanized or veneered form thereof, or a 21F12 antibody or a chimeric, humanized or veneered form thereof.

59. The method of claim 58, wherein the antibody comprises: three light chain variable region complementarity determining regions (CDRs) of 2G3, and three heavy chain variable region CDRs of 2G3.

60. The method of claim 58, wherein the antibody comprises: three light chain variable region CDRs of 14C2, and three heavy chain variable region CDRs of 14C2.

61. The method of claim 58, wherein the antibody comprises: three light chain variable region CDRs, wherein CDR L1 comprises the amino acid sequence of residues 24 to 39 of SEQ ID NO:3, CDR L2 comprises the amino acid sequence of residues 55 to 61 of SEQ ID NO:3, and CDR L3 comprises the amino acid sequence of residues 94 to 102 of SEQ ID NO:3, and three heavy chain variable region CDRs, wherein CDR H1 comprises the amino acid sequence of residues 26 to 35 of SEQ ID NO:2, CDR H2 comprises the amino acid sequence of residues 50 to 66 of SEQ ID NO:2, and CDR H3 comprises the amino acid sequence of residues 99 to 106 of SEQ ID NO:2.

62. The method of any one of claims 49 to 61, wherein the antibody is a chimeric antibody or a humanized antibody.

63. The method of claim 62, wherein the antibody is a humanized antibody.

64. The method of claim 62, wherein the antibody is of the IgG1 subtype.

65. A method of treating a patient diagnosed with Alzheimer's disease that has previously been treated with an antibody with specificity for an N-terminal epitope of Aβ, comprising administering to the patient an effective regime of an antibody that binds to an epitope within residues 12-43 of Aβ and preferentially binds compact plaques relative to diffuse plaques.

66. The method of claim 65, wherein the patient's proportion of compact plaques relative to total plaques increased during prior treatment with the antibody specific for an N-terminal epitope of Aβ.

67. The method of claim 65, wherein the antibody has specificity for a central epitope of Aβ.

68. The method of claim 67, wherein the antibody is a 266 antibody or a chimeric, humanized or veneered form thereof, a 15C11 antibody or a chimeric, humanized or veneered form thereof, or a 22D12 antibody or a chimeric, humanized or veneered form thereof.

69. The method of claim 68, wherein the antibody comprises: three light chain variable region complementarity determining regions (CDRs), wherein CDR L1 comprises the amino acid sequence of SEQ ID NO:4, CDR L2 comprises the amino acid sequence of SEQ ID NO:5, and CDR L3 comprises the amino acid sequence of SEQ ID NO:6, and three heavy chain variable region CDRs, wherein CDR H1 comprises the amino acid sequence of SEQ ID NO:7, CDR H2 comprises the amino acid sequence of SEQ ID NO:8, and CDR H3 comprises the amino acid sequence of SEQ ID NO:9.

70. The method of claim 68, wherein the antibody comprises: three light chain variable region CDRs, wherein CDR L1 comprises the amino acid sequence of residues 24 to 39 of SEQ ID NO:14, CDR L2 comprises the amino acid sequence of residues 55 to 61 of SEQ ID NO:14, and CDR L3 comprises the amino acid sequence of residues 94 to 101 of SEQ ID NO:14, and three heavy chain variable region CDRs, wherein CDR H1 comprises the amino acid sequence of residues 26 to 35 of SEQ ID NO:15, CDR H2 comprises the amino acid sequence of residues 50 to 66 SEQ ID NO:15, and CDR H3 comprises the amino acid sequence of residues 99 to 101 of SEQ ID NO:15.

71. The method of claim 68, wherein the antibody comprises: three light chain variable region CDRs of 22D12, and three heavy chain variable region CDRs of 22D12.

72. The method of claim 65, wherein the antibody has specificity for a C-terminal epitope of Aβ.

73. The method of claim 72, wherein the antibody is a 2G3 antibody or a chimeric, humanized or veneered form thereof, a 14C2 antibody or a chimeric, humanized or veneered form thereof, or a 21F12 antibody or a chimeric, humanized or veneered form thereof.

74. The method of claim 73, wherein the antibody comprises: three light chain variable region complementarity determining regions (CDRs) of 2G3, and three heavy chain variable region CDRs of 2G3.

75. The method of claim 73, wherein the antibody comprises: three light chain variable region CDRs of 14C2, and three heavy chain variable region CDRs of 14C2.

76. The method of claim 73, wherein the antibody comprises: three light chain variable region CDRs, wherein CDR L1 comprises the amino acid sequence of residues 24 to 39 of SEQ ID NO:3, CDR L2 comprises the amino acid sequence of residues 55 to 61 of SEQ ID NO:3, and CDR L3 comprises the amino acid sequence of residues 94 to 102 of SEQ ID NO:3, and three heavy chain variable region CDRs, wherein CDR H1 comprises the amino acid sequence of residues 26 to 35 of SEQ ID NO:2, CDR H2 comprises the amino acid sequence of residues 50 to 66 of SEQ ID NO:2, and CDR H3 comprises the amino acid sequence of residues 99 to 106 of SEQ ID NO:2.

77. The method of any one of claims 65 to 76, wherein the antibody is a chimeric antibody or a humanized antibody.

78. The method of claim 77, wherein the antibody is a humanized antibody.

79. The method of claim 77, wherein the antibody is of the IgG1 subtype.

80. A method of treating a patient diagnosed with Alzheimer's disease that has previously been treated with an antibody that binds to an epitope within residues 12-43 of Aβ and preferentially binds compact plaques relative to diffuse plaques, comprising administering to the patient an effective regime of an antibody with specificity for an N-terminal epitope of Aβ.

81. The method of claim 80, wherein the patient's proportion of diffuse plaques relative to total plaques increased during prior treatment with the antibody specific for a central or C-terminal epitope of Aβ.

82. The method of claim 80, wherein the antibody has specificity for a central epitope of Aβ.

83. The method of claim 82, wherein the antibody is a 266 antibody or a chimeric, humanized or veneered form thereof, a 15C11 antibody or a chimeric, humanized or veneered form thereof, or a 22D12 antibody or a chimeric, humanized or veneered form thereof.

84. The method of claim 83, wherein the antibody comprises: three light chain variable region complementarity determining regions (CDRs), wherein CDR L1 comprises the amino acid sequence of SEQ ID NO:4, CDR L2 comprises the amino acid sequence of SEQ ID NO:5, and CDR L3 comprises the amino acid sequence of SEQ ID NO:6, and three heavy chain variable region CDRs, wherein CDR H1 comprises the amino acid sequence of SEQ ID NO:7, CDR H2 comprises the amino acid sequence of SEQ ID NO:8, and CDR H3 comprises the amino acid sequence of SEQ ID NO:9.

85. The method of claim 83, wherein the antibody comprises: three light chain variable region CDRs, wherein CDR L1 comprises the amino acid sequence of residues 24 to 39 of SEQ ID NO:14, CDR L2 comprises the amino acid sequence of residues 55 to 61 of SEQ ID NO:14, and CDR L3 comprises the amino acid sequence of residues 94 to 101 of SEQ ID NO:14, and three heavy chain variable region CDRs, wherein CDR H1 comprises the amino acid sequence of residues 26 to 35 of SEQ ID NO:15, CDR H2 comprises the amino acid sequence of residues 50 to 66 SEQ ID NO:15, and CDR H3 comprises the amino acid sequence of residues 99 to 101 of SEQ ID NO:15.

86. The method of claim 83, wherein the antibody comprises: three light chain variable region CDRs of 22D12, and three heavy chain variable region CDRs of 22D12.

87. The method of claim 80, wherein the antibody has specificity for a C-terminal epitope of Aβ.

88. The method of claim 87, wherein the antibody is a 2G3 antibody or a chimeric, humanized or veneered form thereof, a 14C2 antibody or a chimeric, humanized or veneered form thereof, or a 21F12 antibody or a chimeric, humanized or veneered form thereof.

89. The method of claim 88, wherein the antibody comprises: three light chain variable region complementarity determining regions (CDRs) of 2G3, and three heavy chain variable region CDRs of 2G3.

90. The method of claim 88, wherein the antibody comprises: three light chain variable region CDRs of 14C2, and three heavy chain variable region CDRs of 14C2.

91. The method of claim 88, wherein the antibody comprises: three light chain variable region CDRs, wherein CDR L1 comprises the amino acid sequence of residues 24 to 39 of SEQ ID NO:3, CDR L2 comprises the amino acid sequence of residues 55 to 61 of SEQ ID NO:3, and CDR L3 comprises the amino acid sequence of residues 94 to 102 of SEQ ID NO:3, and three heavy chain variable region CDRs, wherein CDR H1 comprises the amino acid sequence of residues 26 to 35 of SEQ ID NO:2, CDR H2 comprises the amino acid sequence of residues 50 to 66 of SEQ ID NO:2, and CDR H3 comprises the amino acid sequence of residues 99 to 106 of SEQ ID NO:2.

92. The method of any one of claims 80 to 91, wherein the antibody is a chimeric antibody or a humanized antibody.

93. The method of claim 92, wherein the antibody is a humanized antibody.

94. The method of claim 92, wherein the antibody is of the IgG1 subtype.

95. A method of treating a patient diagnosed with Alzheimer's disease, comprising:
(a) administering to the patient an effective regime of a first antibody that binds to an epitope within residues 12-43 of Aβ and preferentially binds compact plaques relative to diffuse plaques; and
(b) administering to the patient an effective regime of a second antibody with specificity for an N-terminal epitope of Aβ.

96. The method of claim 95, wherein the first and second antibodies are administered concurrently.

97. The method of claim 95, wherein the second antibody is selected from a 3D6 antibody, a 12A11 antibody, a 10D5 antibody, a 12B4 antibody, a 6C6 antibody, a 2H3 antibody, or a 3A3 antibody, or a chimeric, humanized or veneered form of any one of these antibodies.

98. The method of claim 95, wherein the antibody has specificity for a central epitope of Aβ.

99. The method of claim 98, wherein the antibody is a 266 antibody or a chimeric, humanized or veneered form thereof, a 15C11 antibody or a chimeric, humanized or veneered form thereof, or a 22D12 antibody or a chimeric, humanized or veneered form thereof.

100. The method of claim 99, wherein the antibody comprises: three light chain variable region complementarity determining regions (CDRs), wherein CDR L1 comprises the amino acid sequence of SEQ ID NO:4, CDR L2 comprises the amino acid sequence of SEQ ID NO:5, and CDR L3 comprises the amino acid sequence of SEQ ID NO:6, and three heavy chain variable region CDRs, wherein CDR H1 comprises the amino acid sequence of SEQ ID NO:7, CDR H2 comprises the amino acid sequence of SEQ ID NO:8, and CDR H3 comprises the amino acid sequence of SEQ ID NO:9.

101. The method of claim 99, wherein the antibody comprises: three light chain variable region CDRs, wherein CDR L1 comprises the amino acid sequence of residues 24 to 39 of SEQ ID NO:14, CDR L2 comprises the amino acid sequence of residues 55 to 61 of SEQ ID NO:14, and CDR L3 comprises the amino acid sequence of residues 94 to 101 of SEQ ID NO:14, and three heavy chain variable region CDRs, wherein CDR H1 comprises the amino acid sequence of residues 26 to 35 of SEQ ID NO:15, CDR H2 comprises the amino acid sequence of residues 50 to 66 SEQ ID NO:15, and CDR H3 comprises the amino acid sequence of residues 99 to 101 of SEQ ID NO:15.

102. The method of claim 99, wherein the antibody comprises: three light chain variable region CDRs of 22D12, and three heavy chain variable region CDRs of 22D12.

103. The method of claim 95, wherein the antibody has specificity for a C-terminal epitope of Aβ.

104. The method of claim 103, wherein the antibody is a 2G3 antibody or a chimeric, humanized or veneered form thereof, a 14C2 antibody or a chimeric, humanized or veneered form thereof, or a 21F12 antibody or a chimeric, humanized or veneered form thereof.

105. The method of claim 104, wherein the antibody comprises: three light chain variable region complementarity determining regions (CDRs) of 2G3, and three heavy chain variable region CDRs of 2G3.

106. The method of claim 104, wherein the antibody comprises: three light chain variable region CDRs of 14C2, and three heavy chain variable region CDRs of 14C2.

107. The method of claim 104, wherein the antibody comprises: three light chain variable region CDRs, wherein CDR L1 comprises the amino acid sequence of residues 24 to 39 of SEQ ID NO:3, CDR L2 comprises the amino acid sequence of residues 55 to 61 of SEQ ID NO:3, and CDR L3 comprises the amino acid sequence of residues 94 to 102 of SEQ ID NO:3, and three heavy chain variable region CDRs, wherein CDR H1 comprises the amino acid sequence of residues 26 to 35 of SEQ ID NO:2, CDR H2 comprises the amino acid sequence of residues 50 to 66 of SEQ ID NO:2, and CDR H3 comprises the amino acid sequence of residues 99 to 106 of SEQ ID NO:2.

108. The method of any one of claims 95 to 107, wherein the antibody is a chimeric antibody or a humanized antibody.

109. The method of claim 108, wherein the antibody is a humanized antibody.

110. The method of claim 108, wherein the antibody is of the IgG1 subtype.

111. A humanized, chimeric or veneered form of an antibody designated 2G3, 14C2, 21F12, or 22D12.

112. The antibody of claim 111 comprising six Kabat CDRs of the 2G3, 14C2, 21F12 or 22D12 antibody.

113. A method of treating a patient diagnosed with Alzheimer's disease and having a greater proportion of compact plaques than diffuse plaques relative to total plaques, comprising administering to the patient an effective regime of an antibody that binds to an epitope within residues 1-11 of Aβ.

114. The method of claim 113, wherein the proportion of compact plaques is at least 40% of total plaques.

115. The method of claim 113, wherein the proportions of compact and diffuse plaques relative to total plaques are determined by positron emission tomography (PET) scanning.

116. A method of treating a patient diagnosed with Alzheimer's disease and having an MMSE of 1-9 or Braak of 6-7, comprising administering to the patient an effective regime of an antibody that binds to an epitope within residues 12-43 of Aβ and preferentially binds compact plaques relative to diffuse plaques.


SEQUENCE LISTING (21 sequences)

SEQ ID NO: 1 - protein, 42 aa, Homo sapiens [sequence omitted]

SEQ ID NO: 2 - protein, 136 aa, Artificial Sequence
  Description of Artificial Sequence Synthetic polypeptide
MGWNWIFLFLLSGTAGVLSEVQLQQSGPELLKPGASVKISCKASGFTFTDYTMHWMKQSHGKSLEWIGGINPNSGGTIYNEKFKDKATLTVDKSSRTAYMELRSLTSEDSAVYFCTRGVYDGYFYWGQGTLVTVSA

SEQ ID NO: 3 - protein, 131 aa, Artificial Sequence
  Description of Artificial Sequence Synthetic polypeptide
MKLPVRLLVLMFWIPASSSDVVMTQTPLSLPVSLGDQASISCRSSQSLVYSNGNTFLHWYLQKPGQSPKLLIYKVSTRFSGVPDRFSGSGSGSDFTLKISRVEAEDLGIYFCSQTTHAPFTFGSGTKLAIR

SEQ ID NO: 4 - protein, 16 aa, Artificial Sequence
  Description of Artificial Sequence Synthetic peptide
RSSQSLIYSDGNAYLH

SEQ ID NO: 5 - protein, 7 aa, Artificial Sequence
  Description of Artificial Sequence Synthetic peptide
KVSNRFS

SEQ ID NO: 6 - protein, 9 aa, Artificial Sequence
  Description of Artificial Sequence Synthetic peptide
SQSTHVPWT

SEQ ID NO: 7 - protein, 5 aa, Artificial Sequence
  Description of Artificial Sequence Synthetic peptide
RYSMS

SEQ ID NO: 8 - protein, 16 aa, Artificial Sequence
  Description of Artificial Sequence Synthetic peptide
QINSVGNSTYYPDTVK

SEQ ID NO: 9 - protein, 3 aa, Artificial Sequence
  Description of Artificial Sequence Synthetic peptide
GDY

SEQ ID NO: 10 - protein, 113 aa, Artificial Sequence
  Description of Artificial Sequence Synthetic polypeptide
DXVMTQXPLSLPVXXGQPASISCRSSQSLXYSDGNAYLHWFLQKPGQSPXLLIYKVSNRFSGVPDRFSGSGSGTDFTLKISRVEAEDXGVYYCSQSTHVPWTFGXGTXXEIKR

SEQ ID NO: 11 - protein, 112 aa, Artificial Sequence
  Description of Artificial Sequence Synthetic polypeptide
XVQLVEXGGGLVQPGGSLRLSCAASGFTFSRYSMSWVRQAPGKGLXLVAQINSVGNSTYYPDXVKGRFTISRDNXXNTLYLQMNSLRAXDTAVYYCASGDYWGQGTXVTVSS

SEQ ID NO: 12 - protein, 219 aa, Artificial Sequence
  Description of Artificial Sequence Synthetic polypeptide
DVVMTQSPLSLPVTLGQPASISCRSSQSLIYSDGNAYLHWFLQKPGQSPRLLIYKVSNRFSGVPDRFSGSGSGTDFTLKISRVEAEDVGVYYCSQSTHVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 442 aa, Artificial Sequence
  Description of Artificial Sequence Synthetic polypeptide
EVQLVESGGGLVQPGGSLRLSCAASGFTFSRYSMSWVRQAPGKGLELVAQINSVGNSTYYPDTVKGRFTISRDNAKNTLYLQMNSLRAEDTAVYYCASGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 111 aa, Artificial Sequence
  Description of Artificial Sequence Synthetic polypeptide
DVVMTQTPLSLPVSLGDQASISCRSSQSLVHSDGNTYLHWYLQKPGQSPKLLIYKVSNRFSGVPDRFSGSGSGTDFTLKISRVEAEDLGVYFCSQSTHVWTFGGGTKLEIK

SEQ ID NO: 15 - protein, 112 aa, Artificial Sequence
  Description of Artificial Sequence Synthetic polypeptide
EVKLVESGGGLVQPGGSLKLSCAASGFTFSRYSMSWVRQTPEKRLELVAKISNSGDNTYYPDTLKGRFTISRDNAQNTLYLQMSSLKSEDTAMYYCASGDYWGQGTTLTVSS

SEQ ID NO: 16 - protein, 112 aa, Artificial Sequence [sequence omitted]

SEQ ID NO: 17 - protein, 119 aa, Artificial Sequence [sequence omitted]

SEQ ID NO: 18 - protein, 112 aa, Artificial Sequence
  Description of Artificial Sequence Synthetic polypeptide
DVLMTQTPLSLPVSLGDQASISCRSSQNIIHSNGNTYLEWYLQKPGQSPKLLIYKVSNRFSGVPDRFSGSGSGTDFTLKIKKVEAEDLGIYYCFQGSHVPLTFGAGTKLELE

SEQ ID NO: 19 - protein, 123 aa, Artificial Sequence
  Description of Artificial Sequence Synthetic polypeptide
QATLKESGPGILQSSQTLSLTCSFSGFSLSTSGMGVSWIRQPSGKGLEWLAHIYWDDDKRYNPSLKSRLTISKDTSRKQVFLKITSVDPADTATYYCVRRPITPVLVDAMDYWGQGTSVTVSS

SEQ ID NO: 20 - protein, 112 aa, Artificial Sequence
  Description of Artificial Sequence Synthetic polypeptide
DVVMTQSPLSLPVTPGEPASISCRSSQSIVHSNGNTYLEWYLQKPGQSPQLLIYKVSNRFSGVPDRFSGSGSGTDFTLKISRVEAEDVGVYYCFQSSHVPLTFGQGTKLEIK

SEQ ID NO: 21 - protein, 120 aa, Artificial Sequence
  Description of Artificial Sequence Synthetic polypeptide
QVQLVESGGGVVQPGRSLRLSCAFSGFSLSTSGMSVGWIRQAPGKGLEWLAHIWWDDDKYYNPSLKSRLTISKDTSKNTVYLQMNSLRAEDTAVYYCARRTTTADYFAYWGQGTTVTVSS